(19) United States
(12) Patent Application Publication
(10) Pub. No.: US 2008/0008673 A1
(43) Pub. Date: Jan. 10, 2008

(54) Compositions comprising at least one C-glycoside derivative

(76) Inventors: Claudie Willemin, Paris (FR); Laure Bissey-Beugras, Levallois-Perret (FR); Jerome Senee, Lardy (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/822,060

(22) Filed: Jul. 2, 2007

Related U.S. Application Data
(60) Provisional application No. 60/817,683, filed on Jul. 3, 2006.

(51) Int. Cl. A61K 8/97; A61K 8/73

(52) U.S. Cl. 424/70.13; 424/74

(57) ABSTRACT

The present disclosure relates to the combination of at least one C-glycoside derivative with at least one additional cosmetic or dermatological ingredient and/or active agent. Disclosed herein are cosmetic and/or dermatological compositions containing such a combination in a physiologically acceptable medium, or alternatively, a cosmetic or dermatological assembly comprising at least two separate compositions respectively containing for one the C-glycoside derivative and for the other the cosmetic or dermatological ingredient and/or active agent. Also disclosed is a cosmetic treatment method for caring for, cleansing and/or making up the skin and/or its appendages using the combination or the composition(s) according to the invention.

This application claims benefit of U.S. Provisional Application No. 60/817,683, filed Jul. 3, 2006, the contents of which are incorporated herein by reference.

The present invention relates, in particular, to:
the combination of at least one C-glycoside derivative with at least one additional cosmetic or dermatological ingredient and/or active agent;
the cosmetic and/or dermatological compositions containing said combination in a physiologically acceptable medium, or alternatively, a cosmetic or dermatological assembly comprising at least two separate compositions respectively containing for one the C-glycoside derivative and for the other said cosmetic or dermatological ingredient and/or active agent;
the cosmetic or dermatological uses of said combination and/or of said composition containing it; and
a cosmetic treatment method for caring for, cleansing and/or making up the skin and/or its appendages using the combination or the composition(s) according to the invention.

The term “skin and/or its appendages” is understood to mean, in particular, the skin, mucous membranes, lips, scalp, eyelashes, eyebrows and hair.

The cosmetic uses according to the invention comprise especially the improvement of the aesthetic appearance of the skin and/or its appendages, in particular improvement in the surface appearance and/or texture of the skin.

The term “surface appearance” is understood to mean, in particular, the visual and/or tactile irregularities in the skin and/or in the scalp, in particular wrinkles and fine lines, heterogeneity of the skin tone (liver spots, actinic lentigos), defects in the skin microrelief such as chicken pox or acne scars, imperfections of greasy skin (shiny appearance, etc.).

The term “skin texture” is understood to mean, especially, slack, flabby, less firm, less elastic skin, skin that has sagged.

The dermatological uses according to the invention cover the treatment of skin disorders linked to a change in the skin desquamation and/or pigmentation.

Desquamation disorders are understood, in particular, to mean: xerosis, acne, hyperkeratoses, psoriasis, atopy and ichtyosis.

As pigmentation disorders, mention may especially be made of: melasma of the forearms, idiopathic melasmas, hyperpigmentations associated with pregnancy or with oestrogen-progestogen contraception, PUVA lentigines, accidental hyperpigmentation, hyperpigmentation due to leucoderma, and vitiligo.

The combination according to the invention could be formulated in one and the same composition or in two separate compositions, possibly being applied to the skin either simultaneously or successively or after a time delay.

I—C-Glycoside Derivatives

The C-glycoside derivatives used in the invention are, in particular, described in document WO 02/051828.

A C-glycoside derivative suitable for the invention may be a compound of general formula (I) below: 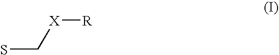
in which:
R represents:
   a saturated C₁ to C₂₀, in particular C₁ to C₁₀, or unsaturated C₂ to C₂₀, in particular C₂ to C₁₀, linear alkyl radical, or a saturated or unsaturated C₃ to C₂₀, in particular C₃ to C₁₀, branched or cyclic alkyl radical; or
   a saturated C₁ to C₂₀, in particular C₁ to C₁₀, or unsaturated C₂ to C₂₀, in particular C₂ to C₁₀, hydrofluoroalkyl or perfluoroalkyl radical, or a saturated or unsaturated C₃ to C₂₀, in particular C₃ to C₁₀, branched or cyclic hydrofluoroalkyl or perfluoroalkyl radical;
      the hydrocarbon chain making up said radicals possibly, where appropriate, being interrupted by 1, 2, 3 or more heteroatoms chosen from:
oxygen;
sulphur;
nitrogen; and
silicon,
   and which may optionally be substituted by at least one radical chosen from:
—OR₄—;
—SR₄;
—NR₄R₅;
—COOR₄;
—CONHR₄;
—CN;
a halogen atom;
a C₁ to C₆ hydrofluoroalkyl or perfluoroalkyl radical;
a C₃ to C₈ cycloalkyl radical; and/or
   with R₄ and R₅ possibly representing, independently of one another, a hydrogen atom, or a saturated C₁ to C₃₀, especially C₁ to C₁₂, or unsaturated C₂ to C₃₀, especially C₂ to C₁₂, linear alkyl, perfluoroalkyl or hydrofluoroalkyl radical, or a saturated or unsaturated C₃ to C₃₀, especially C₃ to C₁₂, branched or cyclic alkyl, perfluoroalkyl or hydrofluoroalkyl, or a C₆ to C₁₀ aryl radical,
X represents a radical chosen from the groups: 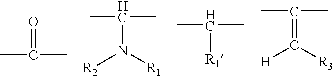
   with R₁, R₂ and R₃ representing, independently of one another, a hydrogen atom, or a radical R, with R as defined previously, and R′₁ represents a hydrogen atom, an —OH group or a radical R as defined previously, R₁ possibly also denoting a C₆ to C₁₀ aryl radical;
S represents a monosaccharide or a polysaccharide composed of up to 20 sugar units, in particular up to 6 sugar units, in the form of pyranose and/or furanose and of L and/or D series, said monosaccharide or polysaccharide possibly being substituted by a compulsorily free hydroxyl group, and optionally one or more optionally protected amine functional groups; and
the S—CH₂—X linkage represents an anomeric C-type linkage, which may be α or β, and also their cosmetically acceptable salts, their solvates such as hydrates and their isomers.

Within the scope of the present invention, the term “halogen” is understood to mean chlorine, fluorine, bromine or iodine.

The term “aryl” denotes an aromatic ring such as a phenyl ring, optionally substituted by one or more C₁-C₄ alkyl radicals.

The term “C₃ to C₈ cycloalkyl” denotes an aliphatic ring having from 3 to 8 carbon atoms, including for example cyclopropyl, cyclopentyl and cyclohexyl rings.

Among the alkyl groups that are suitable for use in the invention, mention may especially be made of methyl, ethyl, isopropyl, n-propyl, n-butyl, t-butyl, isobutyl, sec-butyl, pentyl, n-hexyl, cyclopropyl, cyclopentyl, cyclohexyl and allyl groups.

According to one embodiment of the invention, a C-glycoside derivative corresponding to the formula (I) may be used, for which S may represent a monosaccharide or a polysaccharide containing up to 6 sugar units, in the form of pyranose and/or furanose and of L and/or D series, said monosaccharide or polysaccharide having at least one compulsorily free hydroxyl functional group and/or optionally one or more compulsorily protected amine functional groups, X and R furthermore retaining all the previously given definitions.

Advantageously, a monosaccharide of the invention may be chosen from D-glucose, D-galactose, D-mannose, D-xylose, D-lyxose, L-fucose, L-arabinose, L-rhamnose, D-glucuronic acid, D-galacturonic acid, D-iduronic acid, N-acetyl-D-glucosamine, N-acetyl-D-galactosamine and advantageously denotes D-glucose, D-xylose, N-acetyl-D-glucosamine or L-fucose, and in particular, D-xylose.

More particularly, a polysaccharide of the invention containing up to 6 sugar units may be chosen from D-maltose, D-lactose, D-cellobiose, D-maltotriose, a dissacharide combining a uronic acid chosen from D-iduronic acid or D-glucuronic acid with a hexosamine chosen from D-galactosamine, D-glucosamine, N-acetyl-D-galactosamine, N-acetyl-D-glucosamine, an oligosaccharide containing at least one xylose which may advantageously be chosen from xylobiose, methyl-β-xylobioside, xylotriose, xylotetraose, xylopentaose and xylohexaose and especially the xylobiose that is composed of two xylose molecules joined via a 1-4 linkage.

More particularly, S may represent a monosaccharide chosen from D-glucose, D-xylose, L-fucose, D-galactose, D-maltose and especially D-xylose.

According to another embodiment of the invention, C-glycoside derivatives corresponding to the formula (I) may be used, for which X represents a group chosen from —CO—, —CH(OH)—, —CH(NR₁R₂)—, —CH(R)—, in particular —CO—, —CH(OH)—, —CH(NH₂)—, —CH(NHCH₂CH₂CH₂OH)—, —CH(NHPh)-, —CH(CH₃)—, and more particularly a —CO—, —CH(OH)—, —CH(NH₂)— group, and preferentially a —CH(OH)— group, S and R furthermore retaining all the previously given definitions.

According to another embodiment of the invention, a C-glycoside derivative corresponding to the formula (I) may be used, for which R represents a saturated C₁ to C₂₀, in particular C₁ to C₁₀, or unsaturated C₂ to C₂₀, in particular C₂ to C₁₀, linear alkyl radical, or a saturated or unsaturated C₃ to C₂₀, in particular C₃ to C₁₀, branched or cyclic alkyl radical, which is optionally substituted as described previously, S and R furthermore retaining all the previously given definitions. Preferably, R denotes a C₁-C₄, especially C₁-C₃, linear radical, optionally substituted by —OH, —COOH or —COOR″₂, R″₂ being a saturated C₁-C₄ alkyl radical, especially an ethyl radical. Preferentially R denotes an unsubstituted C₁-C₄, especially C₁-C₂ linear alkyl radical, in particular an ethyl radical.

Among the C-glycoside derivatives of formula (I), preferably those for which the following applies are used:

R represents a saturated C₁ to C₂₀, in particular C₁ to C₁₀, or unsaturated C₂ to C₂₀, in particular C₂ to C₁₀, linear alkyl radical, or a saturated or unsaturated C₃ to C₂₀, in particular C₃ to C₁₀, branched or cyclic alkyl radical, which is optionally substituted as described previously;

S represents a monosaccharide as described previously; and

X represents —CO—, —CH(OH)—, —CH(NR₁R₂)—, —CH(R)— as described previously.

Preferably, a C-glycoside derivative of formula (I) is used for which:

R denotes a C₁-C₄, especially C₁-C₃, linear radical, optionally substituted by —OH, —COOH or —COOR″₂, R″₂ being a saturated C₁-C₄ alkyl radical, especially an ethyl radical;

S represents a monosaccharide as described previously; and

X represents a group chosen from —CO—, —CH(OH)—, —CH(NH₂)—, —CH(NHCH₂CH₂CH₂OH)—, —CH(NHPh)-, —CH(CH₃)—, and more particularly a —CO—, —CH(OH)—, or —CH(NH₂)— group, and preferentially a —CH(OH)— group.

Preferentially, a C-glycoside derivative of formula (I) is used for which:

R denotes an unsubstituted C₁-C₄, especially C₁-C₂, linear alkyl radical, in particular an ethyl radical;

S represents a monosaccharide as described previously; especially D-glucose, D-xylose, N-acetyl-D-glucosamine or L-fucose, and in particular D-xylose; and

X represents a group chosen from —CO—, —CH(OH)—, —CH(NH₂)—, and preferentially a —CH(OH)— group.

Acceptable salts for the non-therapeutic use of the compounds described in the present invention comprise the conventional non-toxic salts of said compounds, such as those formed from organic or inorganic acids. By way of example, mention may be made of the salts of mineral acids, such as sulphuric acid, hydrochloric acid, hydrobromic acid, hydroiodic acid, phosphoric acid, and boric acid. Mention may also be made of the salts of organic acids, which may comprise one or more carboxylic, sulphonic or phosphonic acid groups. They may be linear, branched or cyclic aliphatic acids or else aromatic acids. These acids may comprise, in addition, one or more heteroatoms chosen from O and N, for example in the form of hydroxyl groups. Mention may especially be made of propionic acid, acetic acid, terephthalic acid, citric acid and tartric acid.

When the compound of formula (I) comprises an acid group, neutralization of the acid group or groups may be carried out by a mineral base, such as LiOH, NaOH, KOH, Ca(OH)₂, NH₄OH, Mg(OH)₂ or Zn(OH)₂; or by an organic base such as a primary, secondary or tertiary alkylamine, for example triethylamine or butylamine. This primary, secondary or tertiary alkylamine may comprise one or more nitrogen and/or oxygen atoms and may therefore comprise, for example, one or more alcohol functional groups; mention may especially be made of 2-amino-2-methyl-propanol, triethanolamine, dimethylamino-2-propanol and 2-amino-2-(hydroxymethyl)-1,3-propanediol. Mention may also be made of lysine or 3-(dimethylamino)propylamine.

Acceptable solvates for the compounds described in the present invention comprise conventional solvates such as those formed during the last step of preparing said compounds due to the presence of solvents. By way of example, mention may be made of the solvates due to the presence of water or linear or branched alcohols such as ethanol or isopropanol.

Among the C-glycoside derivatives of formula (I), used according to the invention, the following are most particularly considered:
1. C-β-D-xylopyranoside-n-propan-2-one;
2. C-α-D-xylopyranoside-n-propan-2-one;
3. 1-[2-(3-hydroxypropylamino)propyl]-C-β-D-xylopyranose;
4. 1-[2-(3-hydroxypropylamino)propyl]-C-α-D-xylopyranose;
5. C-β-D-xylopyranoside-2-hydroxypropane;
6. C-α-D-xylopyranoside-2-hydroxypropane;
7. C-β-D-xylopyranoside-2-aminopropane;
8. C-α-D-xylopyranoside-2-aminopropane;
9. C-β-D-xylopyranoside-2-(phenylamino)propane;
10. C-α-D-xylopyranoside-2-(phenylamino)propane;
11. Ethyl ester of 3-methyl-4-(C-β-D-xylopyranoside)butyric acid;
12. Ethyl ester of 3-methyl-4-(C-α-D-xylopyranoside)butyric acid;
13. 6-(C-β-D-xylopyranoside)-5-ketohexanoic acid;
14. 6-(C-α-D-xylopyranoside)-5-ketohexanoic acid;
15. 6-(C-β-D-xylopyranoside)-5-hydroxyhexanoic acid;
16. 6-(C-α-D-xylopyranoside)-5-hydroxyhexanoic acid;
17. 6-(C-β-D-xylopyranoside)-5-aminohexanoic acid;
18. 6-(C-α-D-xylopyranoside)-5-aminohexanoic acid;
19. 6-(C-β-D-xylopyranoside)-5-(phenylamino)hexanoic acid;
20. 6-(C-α-D-xylopyranoside)-5-(phenylamino)hexanoic acid;
21. 1-(C-β-D-xylopyranoside)hexane-2,6-diol;
22. 1-(C-α-D-xylopyranoside)hexane-2,6-diol;
23. 5-(C-β-D-xylopyranoside)-4-ketopentanoic acid;
24. 5-(C-α-D-xylopyranoside)-4-ketopentanoic acid;
25. 5-(C-β-D-xylopyranoside)-4-hydroxypentanoic acid;
26. 5-(C-α-D-xylopyranoside)-4-hydroxypentanoic acid;
27. 5-(C-β-D-xylopyranoside)-4-aminopentanoic acid;
28. 5-(C-α-D-xylopyranoside)-4-aminopentanoic acid;
29. 5-(C-β-D-xylopyranoside)-4-(phenylamino)pentanoic acid;
30. 5-(C-α-D-xylopyranoside)-4-(phenylamino)pentanoic acid;
31. 1-(C-β-D-xylopyranoside)pentane-2,5-diol;
32. 1-(C-α-D-xylopyranoside)pentane-2,5-diol;
33. 1-(C-β-D-fucopyranoside)propan-2-one;
34. 1-(C-α-D-fucopyranoside)propan-2-one;
35. 1-(C-β-L-fucopyranoside)propan-2-one;
36. 1-(C-α-L-fucopyranoside)propan-2-one;
37. 1-(C-β-D-fucopyranoside)-2-hydroxypropane;
38. 1-(C-α-D-fucopyranoside)-2-hydroxypropane;
39. 1-(C-β-L-fucopyranoside)-2-hydroxypropane;
40. 1-(C-α-L-fucopyranoside)-2-hydroxypropane;
41. 1-(C-β-D-fucopyranoside)-2-aminopropane;
42. 1-(C-α-D-fucopyranoside)-2-aminopropane;
43. 1-(C-β-L-fucopyranoside)-2-aminopropane;
44. 1-(C-α-L-fucopyranoside)-2-aminopropane;
45. 1-(C-β-D-fucopyranoside)-2-(phenylamino)propane;
46. 1-(C-α-D-fucopyranoside)-2-(phenylamino)propane;
47. 1-(C-β-L-fucopyranoside)-2-(phenylamino)propane;
48. 1-(C-α-L-fucopyranoside)-2-(phenylamino)propane;
49. Ethyl ester of 3-methyl-4-(C-β-D-fucopyranoside)butyric acid;
50. Ethyl ester of 3-methyl-4-(C-α-D-fucopyranoside)butyric acid;
51. Ethyl ester of 3-methyl-4-(C-β-L-fucopyranoside)butyric acid;
52. Ethyl ester of 3-methyl-4-(C-α-L-fucopyranoside)butyric acid;
53. 6-(C-β-D-fucopyranoside)-5-ketohexanoic acid;
54. 6-(C-α-D-fucopyranoside)-5-ketohexanoic acid;
55. 6-(C-β-L-fucopyranoside)-5-ketohexanoic acid;
56. 6-(C-α-L-fucopyranoside)-5-ketohexanoic acid;
57. 6-(C-β-D-fucopyranoside)-5-hydroxyhexanoic acid;
58. 6-(C-α-D-fucopyranoside)-5-hydroxyhexanoic acid;
59. 6-(C-β-L-fucopyranoside)-5-hydroxyhexanoic acid;
60. 6-(C-α-L-fucopyranoside)-5-hydroxyhexanoic acid;
61. 6-(C-β-D-fucopyranoside)-5-aminohexanoic acid;
62. 6-(C-α-D-fucopyranoside)-5-aminohexanoic acid;
63. 6-(C-β-L-fucopyranoside)-5-aminohexanoic acid;
64. 6-(C-α-L-fucopyranoside)-5-aminohexanoic acid;
65. 1-(C-β-D-fucopyranoside)hexane-2,6-diol;
66. 1-(C-α-D-fucopyranoside)hexane-2,6-diol;
67. 1-(C-β-L-fucopyranoside)hexane-2,6-diol;
68. 1-(C-α-L-fucopyranoside)hexane-2,6-diol;
69. 5-(C-β-D-fucopyranoside)-4-ketopentanoic acid;
70. 5-(C-α-D-fucopyranoside)-4-ketopentanoic acid;
71. 5-(C-β-L-fucopyranoside)hexane-2,6-diol)-4-ketopentanoic acid;
72. 5-(C-α-L-fucopyranoside)hexane-2,6-diol)-4-ketopentanoic acid;
73. 5-(C-β-D-fucopyranoside)-4-hydroxypentanoic acid;
74. 5-(C-α-D-fucopyranoside)-4-hydroxypentanoic acid;
75. 5-(C-β-L-fucopyranoside)-4-hydroxypentanoic acid;
76. 5-(C-α-L-fucopyranoside)-4-hydroxypentanoic acid;
77. 5-(C-β-D-fucopyranoside)-4-aminopentanoic acid;
78. 5-(C-α-D-fucopyranoside)-4-aminopentanoic acid;
79. 5-(C-β-L-fucopyranoside)-4-aminopentanoic acid;
80. 5-(C-α-L-fucopyranoside)-4-aminopentanoic acid;
81. 1-(C-β-D-fucopyranoside)pentane-2,5-diol;
82. 1-(C-α-D-fucopyranoside)pentane-2,5-diol;
83. 1-(C-β-L-fucopyranoside)pentane-2,5-diol;
84. 1-(C-α-L-fucopyranoside)pentane-2,5-diol;
85. 1-(C-β-D-glucopyranosyl)-2-hydroxypropane;
86. 1-(C-α-D-glucopyranosyl)-2-hydroxypropane;
87. 1-(C-β-D-glucopyranosyl)-2-aminopropane;
88. 1-(C-α-D-glucopyranosyl)-2-aminopropane;
89. 1-(C-β-D-glucopyranosyl)-2-(phenylamino)propane;
90. 1-(C-α-D-glucopyranosyl)-2-(phenylamino)propane;
91. Ethyl ester of 3-methyl-4-(C-β-D-glucopyranosyl)butyric acid;
92. Ethyl ester of 3-methyl-4-(C-α-D-glucopyranosyl)butyric acid;
93. 6-(C-β-D-glucopyranosyl)-5-ketohexanoic acid;
94. 6-(C-α-D-glucopyranosyl)-5-ketohexanoic acid;
95. 6-(C-β-D-glucopyranosyl)-5-hydroxyhexanoic acid;
96. 6-(C-α-D-glucopyranosyl)-5-hydroxyhexanoic acid;
97. 6-(C-β-D-glucopyranosyl)-5-aminohexanoic acid;
98. 6-(C-α-D-glucopyranosyl)-5-aminohexanoic acid;
99. 6-(C-β-D-glucopyranosyl)-5-(phenylamino)hexanoic acid;
100. 6-(C-α-D-glucopyranosyl)-5-(phenylamino)hexanoic acid;
101. 1-(C-β-D-glucopyranosyl)hexane-2,6-diol;
102. 1-(C-α-D-glucopyranosyl)hexane-2,6-diol;
103. 6-(C-β-D-glucopyranosyl)-5-ketopentanoic acid;
104. 6-(C-α-D-glucopyranosyl)-5-ketopentanoic acid;
105. 6-(C-β-D-glucopyranosyl)-5-hydroxypentanoic acid;
106. 6-(C-α-D-glucopyranosyl)-5-hydroxypentanoic acid;
107. 6-(C-β-D-glucopyranosyl)-5-aminopentanoic acid;
108. 6-(C-α-D-glucopyranosyl)-5-aminopentanoic acid;
109. 6-(C-β-D-glucopyranosyl)-5-(phenylamino)pentanoic acid;
110. 6-(C-α-D-glucopyranosyl)-5-(phenylamino)pentanoic acid;
111. 1-(C-β-D-glucopyranosyl)pentane-2,5-diol;
112. 1-(C-α-D-glucopyranosyl)pentane-2,5-diol;
113. 1-(C-β-D-galactopyranosyl)-2-hydroxypropane;
114. 1-(C-α-D-galactopyranosyl)-2-hydroxypropane;
115. 1-(C-β-D-galactopyranosyl)-2-aminopropane;
116. 1-(C-α-D-galactopyranosyl)-2-aminopropane;
117. 1-(C-β-D-galactopyranosyl)-2-(phenylamino)propane;
118. 1-(C-α-D-galactopyranosyl)-2-(phenylamino)propane;
119. Ethyl ester of 3-methyl-4-(β-D-galactopyranosyl)butyric acid;
120. Ethyl ester of 3-methyl-4-(α-D-galactopyranosyl)butyric acid;
121. 6-(C-β-D-galactopyranosyl)-5-ketohexanoic acid;
122. 6-(C-α-D-galactopyranosyl)-5-ketohexanoic acid;
123. 6-(C-β-D-galactopyranosyl)-5-hydroxyhexanoic acid;
124. 6-(C-α-D-galactopyranosyl)-5-hydroxyhexanoic acid;
125. 6-(C-β-D-galactopyranosyl)-5-aminohexanoic acid;
126. 6-(C-α-D-galactopyranosyl)-5-aminohexanoic acid;
127. 6-(C-β-D-galactopyranosyl)-5-(phenylamino)hexanoic acid;
128. 6-(C-α-D-galactopyranosyl)-5-(phenylamino)hexanoic acid;
129. 1-(C-β-D-galactopyranosyl)hexane-2,6-diol;
130. 1-(C-α-D-galactopyranosyl)-hexane-2,6-diol;
131. 6-(C-β-D-galactopyranosyl)-5-ketopentanoic acid;
132. 6-(C-α-D-galactopyranosyl)-5-ketopentanoic acid;
133. 6-(C-β-D-galactopyranosyl)-5-hydroxypentanoic acid;
134. 6-(C-α-D-galactopyranosyl)-5-hydroxypentanoic acid;
135. 6-(C-β-D-galactopyranosyl)-5-aminopentanoic acid;
136. 6-(C-α-D-galactopyranosyl)-5-aminopentanoic acid;
137. 6-(C-β-D-galactopyranosyl)-5-(phenylamino)pentanoic acid;
138. 6-(C-α-D-galactopyranosyl)-5-(phenylamino)pentanoic acid;
139. 1-(C-β-D-galactopyranosyl)pentane-2,6-diol;
140. 1-(C-α-D-galactopyranosyl)pentane-2,6-diol;
141. 1-(C-β-D-fucofuranosyl)propan-2-one;
142. 1-(C-α-D-fucofuranosyl)propan-2-one;
143. 1-(C-β-L-fucofuranosyl)propan-2-one;
144. 1-(C-α-L-fucofuranosyl)propan-2-one;
145. 3′-(acetamido-C-β-D-glucopyranosyl)propan-2′-one;
146. 3′-(acetamido-C-α-D-glucopyranosyl)propan-2′-one;
147. 1-(acetamido-C-β-D-glucopyranosyl)-2-hydroxylpropane;
148. 1-(acetamido-C-β-D-glucopyranosyl)-2-aminopropane;
149. 1-(acetamido-C-β-D-glucopyranosyl)-2-(phenylamino)propane;
150. 1-(acetamido-C-α-D-glucopyranosyl)-2-(phenylamino)propane;
151. Ethyl ester of 3-methyl-4-(acetamido-C-β-D-glucopyranosyl)butyric acid;
152. Ethyl ester of 3-methyl-4-(acetamido-C-α-D-glucopyranosyl)butyric acid;
153. 6-(acetamido-C-β-D-glucopyranosyl)-5-ketohexanoic acid;
154. 6-(acetamido-C-α-D-glucopyranosyl)-5-ketohexanoic acid;
155. 6-(acetamido-C-β-D-glucopyranosyl)-5-hydroxyhexanoic acid;
156. 6-(acetamido-C-α-D-glucopyranosyl)-5-hydroxyhexanoic acid;
157. 6-(acetamido-C-β-D-glucopyranosyl)-5-aminohexanoic acid;
158. 6-(acetamido-C-α-D-glucopyranosyl)-5-aminohexanoic acid;
159. 6-(acetamido-C-β-D-glucopyranosyl)-5-(phenylamino)hexanoic acid;
160. 6-(acetamido-C-α-D-glucopyranosyl)-5-(phenylamino)hexanoic acid;
161. 1-(acetamido-C-β-D-glucopyranosyl)hexane-2,6-diol;
162. 1-(acetamido-C-α-D-glucopyranosyl)hexane-2,6-diol;
163. 6-(acetamido-C-β-D-glucopyranosyl)-5-ketopentanoic acid;
164. 6-(acetamido-C-α-D-glucopyranosyl)-5-ketopentanoic acid;
165. 6-(acetamido-C-β-D-glucopyranosyl)-5-hydroxypentanoic acid;
166. 6-(acetamido-C-α-D-glucopyranosyl)-5-hydroxypentanoic acid;
167. 6-(acetamido-C-β-D-glucopyranosyl)-5-aminopentanoic acid;
168. 6-(acetamido-C-α-D-glucopyranosyl)-5-aminopentanoic acid;
169. 6-(acetamido-C-β-D-glucopyranosyl)-5-(phenylamino)pentanoic acid;
170. 6-(acetamido-C-α-D-glucopyranosyl)-5-(phenylamino)pentanoic acid;
171. 1-(acetamido-C-β-D-glucopyranosyl)pentane-2,5-diol;
172. 1-(acetamido-C-α-D-glucopyranosyl)pentane-2,5-diol.

As a non-limiting illustration of the C-glycoside derivatives that are more particularly suitable for the invention, mention may especially be made of the following derivatives:
C-β-D-xylopyranoside-n-propan-2-one;
C-α-D-xylopyranoside-n-propan-2-one;
C-β-D-xylopyranoside-2-hydroxypropane;
C-α-D-xylopyranoside-2-hydroxypropane;
1-(C-α-D-fucopyranoside)propan-2-one;
1-(C-α-D-fucopyranoside)propan-2-one;
1-(C-β-L-fucopyranoside)propan-2-one;
1-(C-α-L-fucopyranoside)propan-2-one;
1-(C-β-D-fucopyranoside)-2-hydroxypropane;
1-(C-α-D-fucopyranoside)-2-hydroxypropane;
1-(C-β-L-fucopyranoside)-2-hydroxypropane;
1-(C-α-L-fucopyranoside)-2-hydroxypropane;
1-(C-β-D-glucopyranosyl)-2-hydroxypropane;
1-(C-α-D-glucopyranosyl)-2-hydroxypropane;
1-(C-β-D-galactopyranosyl)-2-hydroxypropane;
1-(C-α-D-galactopyranosyl)-2-hydroxypropane;
1-(C-β-D-fucofuranosyl) propan-2-one;
1-(C-α-D-fucofuranosyl)propan-2-one;
1-(C-β-L-fucofuranosyl)propan-2-one;
1-(C-α-L-fucofuranosyl)propan-2-one;
—(C—β-D-maltopyranoside-n-propan-2-one;
(C-α-D-maltopyranoside-n-propan-2-one;
—(C-β-D-maltopyranoside-2-hydroxypropane; and
(C-α-D-maltopyranoside-2-hydroxypropane, isomers and blends thereof.

According to one embodiment, C-β-D-xylopyranoside-2-hydroxypropane or C-α-D-xylopyranoside-2-hydroxypropane, and best of all C-β-D-xylo-pyranoside-2-hydroxypropane, may advantageously be used for preparing a composition according to the invention.

According to one particular embodiment, the C-glycoside derivative is C-α-D-xylopyranoside-2-hydroxypropane in the form of a 30 wt % solution of active material in a water/propylene glycol (60/40 wt %) mixture such as the product manufactured by Chimex under the trade name “MEXORYL SBB®”.

Of course, according to the invention, a C-glycoside derivative corresponding to the formula (I) may be used alone or as a blend with other C-glycoside derivatives and in any proportion.

A C-glycoside derivative that is suitable for the invention may especially be obtained by the synthesis method described in document WO 02/051828.

The amount of C-glycoside derivative used in a composition according to the invention depends on the desired cosmetic or therapeutic effect, and may therefore vary over a wide range.

A person skilled in the art may easily, based on their general knowledge, determine the appropriate amounts.

A composition according to the invention may comprise a C-glycoside derivative in an amount of around 0.0001% to around 25% by weight of active material relative to the total weight of the composition, and in particular from around 0.001% to around 10% by weight of active material, and more particularly from around 0.05% to around 5% by weight of active material of C-glycoside derivative relative to the total weight of the composition.

II—Cosmetic or Dermatological Ingredients and/or Active Agents

The term “ingredient” is especially understood, according to the invention, to mean an ingredient favouring the solubilization, stabilization and/or activity of said C-glycoside derivative.

The term “ingredient favouring the stabilization of said previously described C-glycoside derivatives” is understood according to the invention to mean, in particular, an ingredient making it possible to (i) either stabilize said C-glycoside derivative, or (ii) stabilize the physiologically acceptable medium in which said C-glycoside derivative is present.

A—Ingredients Favouring the Solubilization of Said C-Glycoside Derivatives

As ingredients favouring the solubilization of said C-glycoside derivatives that can be used in the compositions of the invention, mention may especially be made of:

(i) lipophilic amino acid derivatives, especially such as those described in Patent Application EP 1 269 986, incorporated here by reference.

In particular, it is possible to use, as lipophilic amino acid derivatives, those of formula:
R′₁(CO)N(R′₂)CH(R′₃)CH₂)ₙ(CO)OR′₄
in which:

n is an integer equal to 0, 1 or 2;

R′₁ represents a linear or branched C₅ to C₂₁ alkyl or alkenyl radical;

R′₂ represents a hydrogen atom or a C₁ to C₃ alkyl group;

R′₃ represents a radical chosen from the group formed by a hydrogen atom, a methyl group, an ethyl group and a linear or branched C₃ or C₄ alkyl radical; and

R′₄ represents a linear or branched C₁ to C₁₀ alkyl radical or linear or branched C₂-C₁₀ alkenyl radical or a sterol residue.

A preferred lipophilic amino acid derivative is isopropyl N-lauroyl sarcosinate especially sold by Ajinomoto under the trade name ELDEW SL 205.

(ii) Fatty alcohols comprising from 8 to 26 carbon atoms, such as lauryl, cetyl, myristyl, stearyl, palmityl, oleyl and linoleyl alcohols, 2-octyldodecanol, and mixtures thereof such as cetostearyl alcohol (mixture of cetyl alcohol and stearyl alcohol). Preferably, 2-octyldodecanol will be used, sold under the name EUTANOL G by Cognis or under the name ISOFOL 20 N/F by Sasol.

(iii) Ethers such as dicaprylyl ether (CTFA: dicaprylyl ether); C₁₂-C₁₅ fatty alcohol benzoates (FINSOLV TN by Finetex). In particular, dicaprylyl carbonate will be used, sold under the name CETIOL CC by Cognis.

According to one particular embodiment, to increase the solubilizing effect of the preceding ingredients, it will be possible, in addition, to add liposoluble UV screening agents such as those described in L'Oréal Patent Application EP 1 221 933.

Mention may especially be made of:

(1) salicylic acid derivatives chosen from homomethyl salicylate, 2-ethylhexyl salicylate, triethanolamine salicylate and 4-isopropylbenzyl salicylate;

(2) cinnamic acid derivatives such as isopentyl 4-methoxycinnamate, 2-ethylhexyl 4-methoxycinnamate, methyl diisopropylcinnamate, isoamyl 4-methoxycinnamate and diethanolamine 4-methoxycinnamate;

(3) liquid β,β′-diphenylacrylate derivatives;

(4) para-aminobenzoic acid derivatives such as 2-ethylhexyl p-(dimethylamino)benzoate and glycerol p-aminobenzoate;

(5) dibenzoylmethane derivatives;

(6) liposoluble benzophenone derivatives;

(7) benzotriazole silicones especially described in Patent Application EP-A-0 392 883;

(8) silicon-containing derivatives of N-substituted benzimidazolyl-benzazoles or of benzofuranyl-benzazoles described, in particular, in Patent Application EP-1 028 120; and

(9) mixtures thereof.

Among the aforementioned cinnamic acid derivatives, it is most particularly preferred, according to the present invention, to use 2-ethylhexyl p-methoxycinnamate or octyl methoxycinnamate, sold, in particular, under the trade name PARSOL MCX by Givaudan.

As dibenzoylmethane derivatives, it is preferred, in particular, to use 4-(tert-butyl)-4′-methoxy-dibenzoylmethane, especially the one sold under the trade name PARSOL 1789 by Givaudan.

Among the salicylic acid derivatives, octyl salicylate will preferentially be used, sold, in particular, under the trade name UVINUL O-18 by BASF.

As benzotriazole silicones, it is preferred to use 2-(2H-benzotriazol-2-yl)-4-methyl-6-[2-methyl-3-[1,3,3,3-tetramethyl-1-[(trimethylsilyl)oxy]disiloxanyl]propynyl]phenol.

In addition, the amount of liposoluble UV screening agents advantageously represents from 0.5 wt % to 20 wt %, best of all from 0.1 to 10 wt %, of the total weight of the composition.

The invention therefore relates to a composition comprising at least one C-glycoside derivative and in which the ingredient favouring the solubilization of said C-glycoside derivatives is one chosen from: a lipophilic amino acid derivative, fatty alcohols comprising from 8 to 26 carbon atoms, dicaprylyl ethers, C₁₂-C₁₅ fatty alcohol benzoates and mixtures thereof.

In particular, the ingredient favouring the solubilization of said C-glycoside derivatives is one chosen from: isopropyl N-lauroyl sarcosinate, 2-octyldodecanol, dicaprylyl ether, a C₁₂-C₁₅ fatty alcohol benzoate and mixtures thereof.

It will be possible, in addition, to add a liposoluble UV screening agents chosen from 2-ethylhexyl p-methoxycinnamate or octyl methoxycinnamate, 4-tert-butyl-4′-methoxy-dibenzoylmethane, octyl salicylate and 2-(2H-benzotriazol-2-yl)-4-methyl-6-[2-methyl-3-[1,3,3,3-tetramethyl-1-[(trimethylsilyl)oxy]disiloxanyl]propynyl]phenol.

B—Ingredients Favouring the Stabilization of Said C-Glycoside Derivatives

The term “ingredient favouring the stabilization of said C-glycoside derivatives” is understood according to the invention to mean, in particular, an ingredient making it possible to (i) either stabilize said C-glycoside derivative, or (ii) stabilize the physiologically acceptable medium in which said C-glycoside derivative is present.

In particular, in the case (ii), the ingredient favouring the stabilization of said C-glycoside derivatives may especially be present in a particular galenic form that contributes to the stabilization of said C-glycoside derivative in said physiologically acceptable medium.

As ingredients for stabilizing said C-glycoside derivatives that can be used in the compositions of the invention, mention may especially be made of:
(a) block polymers and/or copolymers;
(b) ionic or non-ionic type amphiphilic lipids present in the form of vesicles in dispersion;
(c) constituent polymers of nanoparticles, in particular of nanospheres or nanocapsules;
(d) constituent polymers of microparticles;
(e) polymers and/or surfactants forming nanoemulsions;
(f) polymers in the form of thin films;
(g) emulsifiers based on polyolefins having a polar part, the composition being in the form of a water-in-oil emulsion; and
(h) amphiphilic polymers comprising 2-acrylamido-2-methylpropane sulphonic acid (AMPS) units.
   (a) Block Polymers and/or Copolymers

It is known how to encapsulate active agents into micelles of block copolymers, for example polyethylene oxide/propylene oxide diblock or triblock copolymers.

Advantageously, an amphiphilic block copolymer comprising at least one non-ionic hydrophilic polymer block and at least one particular hydrophobic polymer block will be used in the composition of the invention.

These amphiphilic block copolymers are especially described in Patent Application EP 1 555 984, incorporated here by reference.

The molecular weight of the block copolymer is generally between 1000 and 100 000.

In particular, the weight ratio of the ionic or non-ionic hydrophilic polymer block(s) to the hydrophobic polymer block(s) is between 1/100 and 50/1.

The hydrophobic polymer block is especially chosen from:
styrene and its derivatives, such as 4-butylstyrene;
alkylene oxides comprising more than 4 carbon atoms, and preferably from 4 to 6 carbon atoms;
hydrophobic vinyl monomers of formula (A) below: 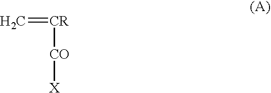
   in which:
R is chosen from H or —CH₃,
X is chosen from —OR′ type alkyl oxides where R′ is a saturated or unsaturated, linear or branched, hydrocarbon-based radical having from 1 to 22 carbon atoms.

Preferably, the hydrophobic polymer block is obtained from one or more hydrophobic monomers chosen from methyl methacrylate, ethyl methacrylate, n-butyl (meth)acrylate, tert-butyl(meth)acrylate and cyclohexyl acrylate.

In particular, the hydrophobic polymer block is chosen from polystyrene, poly(tert-butylstyrene), polymethyl methacrylate, polyethyl acrylate, polybutyl methacrylate, and C₃-C₆ polyalkylene oxides.

Preferably, the non-ionic hydrophilic polymer block is chosen from polyethylene oxides.

Preferably, the block copolymer is chosen from the following block copolymers:

polystyrene/polyoxyethylene;

polymethyl methacrylate/polyoxyethylene;

polybutyl methacrylate/polyoxyethylene; and

polyoxyethylene/polyoxybutylene/polyoxyethylene.

(b) Ionic or Non-Ionic Type Amphiphilic Lipids

According to another embodiment of the invention, the C-glycoside derivatives are combined with ionic or non-ionic amphiphilic lipids present in the form of ionic (e.g. liposomes) and/or non-ionic (e.g. niosomes) vesicles in dispersion in the physiologically acceptable medium of the composition, in particular in an aqueous dispersion.

The presence of these vesicles contributes to the stabilization of said C-glycoside derivatives in the physiologically acceptable medium of the composition.

These lipid vesicles may have an aqueous core or an oily core.

Preferentially, lipid vesicles having an oily core will be used.

The term “vesicle” is understood, according to the invention, to mean any particulate structure comprising, on the one hand, a membrane or “lipid phase” formed from one or more concentric sheets, these sheets comprising one or more bimolecular layers based on ionic or non-ionic amphiphilic lipids and, on the other hand, an aqueous or oily phase encapsulated by this lipid phase. According to the meaning of the invention, the liposomes and niosomes form, in particular, such vesicles.

Niosomes are vesicles prepared from non-ionic amphiphilic lipids. Reference may especially be made to the description of Patent FR 8 907 947, incorporated into the present invention by reference.

As non-ionic amphiphilic lipid, mention may especially be made of optionally oxyethylenated alkyl or polyalkyl esters, and optionally oxyethylenated polyol ethers, having a melting point of at least 40° C.

Liposomes are vesicles prepared from ionic amphiphilic lipids. These vesicles are particles formed from a membrane composed of one or more concentric sheets, these sheets comprising one or more bimolecular layers of amphiphilic lipids encapsulating an aqueous or oily phase. The aqueous phase may contain water-soluble active substances and the bimolecular layers of amphiphilic lipids may contain lipophilic active substances. These vesicles generally have an average diameter between 10 and 5000 nanometres.

The ionic amphiphilic lipids may be anionic amphiphilic lipids or cationic amphiphilic lipids.

As examples of anionic amphiphilic lipids, mention may especially be made of:

preferably, neutralized anionic lipids, chosen from the alkali-metal salts of dicetyl phosphate and of dimyristoyl phosphate, in particular the sodium and potassium salts, the alkali-metal salts of phosphatidic acid, in particular the sodium salt, the alkali-metal salts of cholesterol sulphate, in particular the sodium salt, the alkali-metal salts of cholesterol phosphate, in particular the sodium salt, the salts of lipoamino acids such as monosodium or disodium acylglutamates, more particularly the disodium salt of N-stearoyl-L-glutamic acid sold under the name ACYLGLUTAMATE HS21 by Ajinomoto;

amphoteric lipids, in particular pure soybean phosphatidylethanolamine; and

alkylsulphonic derivatives.

As cationic amphiphilic lipids, quaternary ammonium salts, fatty amines and salts thereof may especially be used.

It is possible to advantageously use “double liposome” compositions for simultaneously treating superficial and deep skin layers, comprising a first dispersion of lipid vesicles capable of penetrating into the deep skin layers and containing at least one active agent capable of treating these deep layers and a second dispersion of lipid vesicles capable of penetrating into the superficial skin layers and containing at least one active agent capable of treating these superficial layers. Such a system is described in Patent EP 0 661 035, incorporated here by reference.

Oleosomes are oily globules provided with a lamellar liquid crystal coating that are dispersed in an aqueous

phase, having an average diameter that is generally less than 500 nanometres.

As an example of a formulation in oleosomes, reference may especially be made to Patent EP 0 641 557, incorporated into the present invention by reference.

(c) Constituent Polymers of Nanoparticles

According to one alternative, it will be possible to combine said C-glycoside derivatives with small-sized particles, in particular known as nanoparticles.

They could be solid particles formed from the combination of said C-glycoside derivatives with at least one polymer.

This polymer contributes towards stabilizing said C-glycoside derivative in the physiologically acceptable medium of the composition.

The term “nanoparticles” mainly encompasses two different systems: “nanospheres” formed from a polymer matrix in which said C-glycoside derivative is absorbed and/or adsorbed and/or mixed, and also “nanocapsules” having a core-shell type structure, that is to say a structure composed of a lipid core that is liquid at room temperature containing the C-glycoside derivative in dissolved form, which core is encapsulated in a continuous protective shell that is insoluble in the medium.

Preferably nanocapsules will be used.

Reference may be made, for example, to the description of Patent Application EP 1 414 390, incorporated here by reference.

The nanospheres generally have an average size between 50 and 500 nm.

The nanocapsules are generally small-sized so as to obtain optimal bioavailability of the C-glycoside derivatives.

Preferentially, the size of these nanocapsules is between 10 nm and 1000 nm and more particularly between 30 nm and 500 nm.

It will especially be possible to use polymers in the form of nanocapsules as described in Patent Application EP 0 274 961, the nanocapsules provided with a lamellar coating described in Application EP 0 780 115, the nanocapsules whose continuous polymer shell, which is insoluble in water, is formed from polyesters, as described in Applications EP 1 025 901, FR 2 787 730 and EP 1 034 839, or else the biodegradable nanocapsules described in Patent Application FR 2 659 554, or the non-biodegradable nanocapsules described in Patent Application WO 93/05753.

The nanocapsules made from biodegradable polymers penetrate into the skin and are degraded in the epidermis under the action of enzymes that are present therein, whereas the nanocapsules made from non-biodegradable polymers only penetrate into the superficial layers of the stratum corneum and are naturally eliminated during skin renewal.

As constituent polymers of nanocapsules that can be used in the compositions of the invention in combination with the C-glycoside derivatives, mention may especially be made of poly-L-lactides and poly-DL-lactides and polycaprolactones, polyglycolides and copolymers thereof, polymers derived from the polymerization of an alkyl cyanoacrylate (the alkyl chain having from 2 to 6 carbon atoms); synthetic or natural water-dispersible anionic polymers; polyesters of the poly(alkylene adipate) type; dendritic polymers; vinyl chloride/vinyl acetate copolymers, copolymers of methacrylic acid and of the methyl ester of methacrylic acid, polyvinyl acetophthalate, cellulose acetophthalate, crosslinked polyvinyl pyrrolidone/vinyl acetate copolymers, polyethylene vinyl acetates, polyacrylonitriles, polyacrylamides, polyethylene glycols, polyamides, polyethylenes, polypropylenes and organopolysiloxanes.

Preferably, polycaprolactones will be used.

(d) Constituent Polymers of Microparticles

According to one particular embodiment of the invention, especially advantageous for the care of greasy skin, the C-glycoside derivatives will be combined with constituent polymers of microparticles.

These polymers contribute towards stabilizing said C-glycoside derivative in the physiologically acceptable medium of the composition.

The term “microparticles” especially encompasses “porous particles” and in particular “microspheres”.

The term “porous particles” is understood to denote particles having a structure comprising pores. This porous structure may, at least partially, make it possible to incorporate one or more active agents within said particles.

Reference may especially be made to Patent Application EP 1 637 124, incorporated here by reference.

The structure of the particles may be of the matrix type similar to a sponge. It may also be of the vesicle type, that is to say that the particle has an internal cavity delimited by a porous wall. The porosity associated with the size of the particles is characterized quantitatively by their specific surface area.

Porous particles will especially be used having a specific surface area measured according to the BET method greater than or equal to 1 m²/g. The BET (Brunauer-Emmet-Teller) method is a method that is well known to a person skilled in the art. It is especially described in “The Journal of the American Chemical Society”, Vol. 60, page 309, February 1938, and corresponds to the International Standard ISO 5794/1 (Annex D). The specific surface area measured according to the BET method corresponds to the total specific surface area, that is to say that it includes the surface formed by the pores.

According to one particular embodiment, the particles of the invention have a specific surface area measured by the BET method, ranging, in particular, from 2.5 to 100 m²/g.

The porous particles that can be used in the compositions of the invention are generally individual particles. The expression “individual particles” denotes particles that are not grouped together in the form of an aggregate or agglomerate. These particles have, in particular, a density greater than or equal to 0.15 g/cm³ and especially ranging from 0.2 to 5 g/cm³.

These particles preferably have a volume-average diameter less than or equal to 10 μm. Indeed, such particles can penetrate into the sebaceous follicle by application of a mechanical force. This mechanical force generally results from a massage which, besides the pressure that it exerts, generates a pump effect at the follicle. The particles thus gradually reach the follicle channel in which they are capable of absorbing sebum and, where appropriate, of releasing the active compound that they carry. The constituent material of the particles is then discharged thanks to the flow of sebum and/or the growth of the hair, thus enabling any possible undesirable reaction of the body towards this material to be prevented.

In particular, particles, especially spherical porous particles having a number-average size which may range from 0.1 to 50 μm, especially from 0.1 to 20 μm and most particularly from 0.5 to 10 μm, will be used.

As preferred porous particles, it is possible to use polyamide particles in particular nylon-6, nylon-6,6, nylon-12 or nylon-6,12 such as those sold by Atofina under their generic name ORGASOL.

This encapsulation system, which allows follicular targeting, is particularly advantageous in compositions intended for treating greasy skin.

(e) Polymers and/or Surfactants Forming Nanoemulsions

According to another embodiment of the invention, said C-glycoside derivatives are combined in the composition with particular polymers and/or surfactants, the composition being in the form of a nanoemulsion.

These polymers and/or surfactants contribute towards stabilizing said C-glycoside derivatives in the physiologically acceptable medium of the composition.

The nanoemulsions are generally oil-in-water emulsions whose oil globules have a very fine particle size distribution, that is to say a number-average size less than 100 nanometres (nm).

Reference may especially be made to the description of Patent EP 0 728 460, incorporated here by reference.

The nanoemulsions may be stabilized by a lamellar liquid crystal coating obtained by combining a hydrophilic surfactant with a lipophilic surfactant.

Advantageously, the particular polymers that make it possible to produce nanoemulsions may be chosen from:

anionic polymers having a hydrophobic chain, as described in Patent EP 116 005, incorporated here by reference; and/or

water-soluble non-ionic polymers as described in Patent EP 1 172 077, incorporated here by reference.

The particular surfactants that make it possible to produce nanoemulsions may, in particular, be a ternary surfactant system including a mixture of non-ionic surfactants and an ionic surfactant, as described in Patent EP 1 353 629, incorporated here by reference.

In particular, the anionic polymer having a hydrophobic chain comprises hydrophobic chains chosen from saturated or unsaturated, linear or branched hydrocarbon-based chains having from 6 to 30 carbon atoms, divalent cycloaliphatic groups and divalent aromatic groups, and preferably chosen from alkyl, arylalkyl, alkylaryl, alkylene, methylenedicyclohexyl, isophorone and phenylene chains.

In particular, the anionic polymer is chosen from copolymers of acrylic or methacrylic acid, copolymers of 2-acrylamido-2-methylpropanesulphonic acid and blends thereof.

Preferably, the anionic polymer is obtained by copolymerizing a monomer (a) chosen from α, β-ethylenically unsaturated carboxylic acids (monomer a′) and 2-acrylamido-2-methylpropanesulphonic acid (monomer a″), with a non-surfactant ethylenically unsaturated monomer (b) different from (a) and/or an ethylenically unsaturated monomer (c) derived from the reaction of an α,β-monoethylenically unsaturated acrylic monomer or a monoethylenically unsaturated isocyanate monomer with a monohydric non-ionic amphiphilic component or with a primary or secondary fatty amine.

Advantageously, the anionic polymer is an acrylic terpolymer obtained from (a) an α,β-ethylenically unsaturated carboxylic acid, (b) a non-surfactant ethylenically unsaturated monomer different from (a) and (c) a non-ionic urethane monomer that is the reaction product of a monohydric non-ionic amphiphilic compound with a monoethylenically unsaturated isocyanate.

For example, the anionic polymer is chosen from the acrylic acid/ethyl acrylate/alkyl acrylate terpolymer, the acrylates/steareth-20 methacrylate copolymer, the oxyethylenated (25 EO) (meth)acrylic acid/ethyl acrylate/behenyl methacrylate terpolymer, the oxyethylenated (20 EO) acrylic acid/monocetyl itaconate copolymer, the oxyethylenated (20 EO) acrylic acid/monostearyl itaconate copolymer, the acrylates/acrylate modified by polyoxyethylenated (25 EO) C₁₂-C₂₄ alcohols copolymer, the ethoxylated methacrylic acid/methyl acrylate/behenyl alcohol dimethyl-meta-isopropenyl benzylisocyanate terpolymer and blends thereof.

In particular, the water-soluble non-ionic polymer may be chosen from ethylene oxide homopolymers and copolymers; polyvinyl alcohols; vinylpyrrolidone homopolymers and copolymers; vinylcaprolactam homopolymers and copolymers; polyvinyl methyl ether homopolymers and copolymers; neutral acrylic homopolymers and copolymers; C₁-C₂ alkyl celluloses and their derivatives; C₁-C₃ alkyl guar or C₁-C₃ hydroxyalkyl guar.

The ternary surfactant system may comprise in particular:

(a) a mixture of at least two non-ionic surfactants comprising at least one ethoxylated fatty ester having 8 to 100 units (especially 10 to 80 units, and best 40 units) of ethylene oxide and at least one sorbitan fatty acid ester; and

(b) at least one ionic surfactant chosen from the alkali-metal salts of cetyl phosphate and the alkali-metal salts of palmitoyl sarcosinate.

The ethoxylated fatty ester is preferably polyethylene glycol (40 EO) stearate and the sorbitan fatty acid ester is preferably sorbitan tristearate.

The ionic surfactant is especially chosen from potassium cetyl phosphate, sodium palmitoyl sarcosinate and mixtures thereof.

(f) Polymers in the Form of Thin Films

According to another embodiment of the invention, said C-glycoside derivatives are combined with at least one water-soluble or water-dispersible polymer in the form of a thin film.

The composition is therefore in the form of a thin film.

This water-soluble or water-dispersible polymer contributes towards stabilizing C-glycoside derivatives in the physiologically acceptable medium of the composition.

The term “film” is understood in the present application to mean a thin solid that can be grasped. The term “thin” is understood to mean a solid having a maximum thickness of 1000 μm. This film generally has a sufficient size to be able to be easily handled by the user. It may be in the shape of a square, rectangle, disc or any other shape. Each film generally has a thickness from 10 μm to 1000 μm, preferably from 20 to 500 μm and best from 50 to 300 μm. It may have a surface area of 0.25 to 25 cm² and preferably from 2 to 10 cm².

These thin films generally contain less than 10% by weight of water, preferably less than 5% by weight relative to the total weight of the film, and more preferably, do not contain any water.

Such films are described in Patent Application EP 1 588 694, incorporated here by reference.

The thin film comprises a water-soluble or water-dispersible polymer which may be chosen from: (1) proteic-type polymers, such as wheat or soybean proteins; keratin, for example keratin hydrolysates and sulphonic keratins; casein; albumin; collagen; glutelin; glucagon; gluton; zein; gelatins and their derivatives; (2) polymers deriving from chitin or chitosan, such as anionic, cationic, amphoteric or non-ionic chitin or chitosan polymers; (3) polysaccharide polymers such as, especially (i) cellulose polymers, such as hydroxyethyl cellulose, hydroxypropyl cellulose, hydroxypropyl methyl cellulose, methyl cellulose, ethyl hydroxyethyl cellulose, carboxymethyl cellulose, quaternary derivatives of cellulose; and (ii) starches and their derivatives; (4) acrylic polymers or copolymers such as polyacrylates, polymethacrylates and copolymers thereof; (5) vinyl polymers such as polyvinylpyrrolidones, methyl vinyl ether/maleic anhydride copolymers, vinyl acetate/crotonic acid copolymer, vinyl-pyrrolidone/vinyl acetate copolymers, vinylpyrrolidone/caprolactam copolymers and polyvinyl alcohols; (6) optionally modified polymers of natural origin, such as gum arabic, guar gum, xanthan derivatives, karaya gum; alginates, carrageenans, ulvans and other algal colloids; glycoaminoglycans, hyaluronic acid and its derivatives; shellac, gum sandarac, dammars, elemi gum, copals; deoxyribonucleic acid; mucopolysaccharides, such as hyaluronic acid, chondroitin sulphate; and blends of these polymers.

Mention may also be made, as water-soluble polymers, of caprolactams, pullulan, pectin, mannan and galactomannans, glucomannans and their derivatives.

Preferably, the water-soluble polymer may be a cellulose polymer, in particular hydroxypropyl cellulose or hydroxypropyl methyl cellulose, or else an alginate, especially sodium alginate.

The composition according to the invention may be in the form of an emulsion and may also contain specific emulsifiers capable of stabilizing the C-glycoside derivatives and may advantageously confer outstanding sensory properties, such as a light and fresh feel.

As examples of particular emulsifiers, mention may especially be made of:
emulsifiers based on polyolefins having a polar part, in a water-in-oil emulsion; and
amphiphilic polymers comprising 2-acrylamido-2-methylpropanesulphonic acid (AMPS) units, in an oil-in-water emulsion.
   G) Emulsifiers Based on Polyolefins Having a Polar Part

The invention therefore also relates to a composition in the form of a water-in-oil emulsion comprising at least one C-glycoside derivative (I) or (II) and at least one polyolefin having a polar part.

The polyolefins having a polar part contribute towards stabilizing said C-glycoside derivative in the physiologically acceptable medium of the composition.

The polyolefins having polar part(s) used in the composition of the invention are generally composed of an apolar polyolefin part and at least one polar part. The apolar polyolefin part comprises at least 40 carbon atoms and preferably from 60 to 700 carbon atoms. This apolar part may be chosen from polyolefins such as oligomers, polymers and/or copolymers of ethylene, propylene, 1-butene, isobutene, 1-pentene, 2-methyl-1-butene, 3-methyl-1-butene, 1-hexene, 1-heptene, 1-octene, 1-decene, 1-undecene, 1-dodecene, 1-tridecene, 1-tetradecene, 1-pentadecene, 1-hexadecene, 1-heptadecene and 1-octadecene. These polyolefins may or may not be hydrogenated.

The polar part of the polyolefins having a polar part may be anionic, cationic, non-ionic, zwitterionic or amphoteric. It is, for example, formed from polyalkylene glycols (especially polyoxyethylene glycols) or from polyalkylene imines, or else from carboxylic acids or diacids, their anhydrides or their derivatives such as their esters, their amides and their salts, and blends thereof. Polyolefins having a carboxylic acid polar part may be, for example, derived from the reaction between a polyolefin and at least one carboxylic acid or anhydride that is optionally completely or partially salified, chosen from the group comprising succinic acid or anhydride, maleic acid, maleic anhydride, fumaric acid, itaconic acid, citraconic acid (or methylmaleic acid), mesaconic acid (or methylfumaric acid), aconitic acid, their ester or amide derivatives, and mixtures thereof.

Preferably, the polar part of the polyolefin is chosen from the group comprising polyoxyethylene, succinic acid or anhydride, the esters or amides of succinic acid or anhydride, the alkali-metal or alkaline-earth-metal salts or organic salts of succinic acid or anhydride, or the partial salts of monoesters or monoamides of succinic acid or anhydride.

The polyolefins having a polar part that are preferred in the compositions of the invention are polyolefins having optionally modified succinic ends, as described in Patent EP 1 172 089, incorporated here by reference.

As polyolefins having succinic ends, mention may especially be made of polyisobutylenes having optionally modified esterified succinic ends, especially esterified by diethanolamine, and their salts, especially diethanolamine salts, such as the products sold under the names LUBRIZOL® 2724, LUBRIZOL® 2722 and LUBRIZOL® 5603 by the company Lubrizol.

Another polyolefin having a polar part that is particularly preferred is an ester of triethanolamine-diethanolaminoethyl polyisobutenyl succinate. This product is sold, for example, under the name CHEMCCINATE® 2000 by Chemron.

As the polyolefin having a polar part, it is also possible to use an ester of glyceryl polyisobutenyl succinate, especially that sold under the name CHEMCCINATE® 1000 AF by Chemron.

h) Amphiphilic Polymers Comprising AMPS Units

According to another embodiment of the invention, the composition according to the invention is in the form of an oil-in-water emulsion and comprises at least one C-glycoside derivative and at least one amphiphilic polymer comprising 2-acrylamido-2-methyl-propanesulphonic acid (AMPS) units.

This amphiphilic polymer comprising 2-acrylamido-2-methylpropanesulphonic acid (AMPS) units contributes towards stabilizing said C-glycoside derivative in the physiologically acceptable medium of the composition.

As the amphiphilic polymer comprising 2-acrylamido-2-methylpropanesulphonic acid (AMPS) units that can be used in the compositions of the invention, mention may be made of those described in Patent Application EP 1 466 587, incorporated here by reference.

As preferred amphiphilic polymers, mention may be made of the copolymers of AMPS and oxyethylenated C₁₂-C₁₄ alcohol methacrylate, especially comprising from 7 to 23 oxyethylenated groups.

To improve the sensory properties of the composition, such as a fresh effect and a feel that is neither sticky nor greasy, in particular for oil-in-water emulsions, tetrapolymers may also be used.

The tetrapolymer used according to the invention comprises, as monomers, methacrylic acid, methylmethacrylate, butylacrylate and a C₁₆-C₂₀ alkyl(meth)acrylate.

Tetrapolymers as defined previously, and also their preparation method, are in particular described in Application US 2003/0021847.

These tetrapolymers may be prepared by emulsion polymerization of the monomers indicated above in the presence of a free-radical initiator such as hydrogen peroxide, tert-butyl hydroperoxide, or sodium, potassium, lithium or ammonium persulphate, the initiator optionally being combined with a reducing agent in order to form a redox system and with a catalyst composed of a transition metal such as a copper or iron salt. The reaction may, for example, be carried out at a temperature between 10 and 120° C., preferably around 85° C., for an approximate duration of three hours.

A tetrapolymer of this type is in particular available from Rohm & Haas under the trade name ALLIANZ OPT in the form of a hydroglycolic dispersion having 48% of active material.

The amount of tetrapolymer (as active material) in the composition according to the invention may range, for example, from 0.1 to 5 wt %, preferably from 0.2 to 5 wt %, better from 0.2 to 2 wt %, and better still from 0.5 to 1 wt % of tetrapolymer, relative to the total weight of the composition.

The compositions according to the invention as defined previously and according to the chosen embodiment (choice of ingredient favouring the solubilization and/or stabilization of said C-glycoside derivatives), may be in any galenic form conventionally used for a topical application and especially in the form of aqueous or aqueous-alcoholic solutions, oil-in-water (O/W) or water-in-oil (W/O) or multiple (triple: W/O/W or O/W/O) emulsions, aqueous gels, or dispersions of a fatty phase in an aqueous phase using polymeric nanoparticles such as nanospheres and nanocapsules, or ionic and/or non-ionic type lipid vesicles (liposomes, niosomes, oleosomes), nanoemulsions, or thin films. It is also possible to use a composition according to the invention in the form of a dual water and oil phase.

These compositions are prepared according to the normal methods.

In addition, the compositions used according to the invention may be more or less liquid and have the appearance of a white or coloured cream, an ointment, a milk, a lotion, a serum, a paste or a mousse. They may optionally be applied to the skin in the form of an aerosol. They may also be in solid form, and for example in stick form.

When the composition used according to the invention comprises an oily phase, this preferably contains at least one oil. It may contain, in addition, other fatty substances.

As the oils that can be used in the composition of the invention, mention may, for example, be made of:

hydrocarbon-based oils of animal origin, such as perhydrosqualene;

hydrocarbon-based oils of plant origin, such as liquid triglycerides of fatty acids comprising from 4 to 10 carbon atoms such as heptanoic or octanoic acid triglycerides or else, for example sunflower, maize, soybean, pumpkin, grape seed, sesame, hazelnut, apricot, macadamia, arara, sunflower, caster oil and avocado oils, caprylic/capric acid triglycerides such as those sold by Stearineries Dubois or those sold under the names MIGLYOL 810, 812 and 818 by Dynamit Nobel, jojoba oil, shea butter oil;

synthetic esters and ethers, especially of fatty acids, such as oils of formulae R¹COOR² and R¹OR² in which R¹ represents the residue of a fatty acid comprising 8 to 29 carbon atoms, and R² represents a branched or unbranched hydrocarbon-based chain containing 3 to 30 carbon atoms, such as for example purcellin oil, isononyl isononanoate, isopropyl myristate, 2-ethylhexyl palmitate, 2-octyldodecyl stearate, 2-octyldodecyl erucate, isostearyl isostearate; hydroxylated esters such as isostearyl lactate, octyl hydroxystearate, octyidodecyl hydroxystearate, diisostearyl malate, triisocetyl citrate, heptanoates, octanoates and decanoates of fatty alcohols; polyol esters, such as propylene glycol dioctanoate, neopentyl glycol diheptanoate and diethylene glycol diisononanoate; and pentaerythritol esters such as pentaerythrityl tetraisostearate;

linear or branched hydrocarbons, of mineral or synthetic origin, such as volatile or non-volatile paraffin oils and their derivatives, petroleum jelly, polydecenes, hydrogenated polyisobutene such as parleam oil;

fatty alcohols having from 8 to 26 carbon atoms, such as cetyl alcohol, stearyl alcohol and their blend (cetostearyl alcohol), octyldodecanol, 2-butyloctanol, 2-hexyldecanol, 2-undecylpentadecanol, oleyl alcohol or linoleyl alcohol;

partially hydrocarbon-based and/or silicone-based fluoro oils such as those described in document JP-A-2 295 912;

silicone oils such as volatile or non-volatile polydimethylsiloxanes (PDMS) having linear or cyclic silicone-based chains that are liquid or pasty at room temperature, especially cyclopolydimethylsiloxanes (cyclomethicones) such as cyclohexasiloxane; polydimethyl-siloxanes comprising alkyl, alkoxy or phenyl groups, which are pendent or at the end of the silicone-based chain, groups having 2 to 24 carbon atoms; phenylsilicones such as phenyltrimethicones, phenyldimethicones, phenyltrimethylsiloxydiphenylsiloxanes, diphenyldimethicones, diphenylmethyldiphenyltrisiloxanes, 2-phenylethyltrimethylsiloxysilicates, and polymethylphenylsiloxanes; and

mixtures thereof.

The term “hydrocarbon-based oil” in the aforementioned list of oils is understood to mean any oil composed mainly of carbon and hydrogen atoms, and optionally of ester, ether, fluoro, carboxylic acid and/or alcohol groups.

The other fatty substances which may be present in the oily phase are, for example, fatty acids comprising from 8 to 30 carbon atoms, such as stearic acid, lauric acid, palmitic acid and oleic acid; waxes such as lanolin, beeswax, carnauba or candelilla wax, paraffin and montan waxes or microcrystalline waxes, ceresin or ozokerite, synthetic waxes such as polyethylene waxes, Fischer-Tropsch waxes; silicone resins such as trifluoromethyl-C₁₋₄-alkyldimethicone and trifluoropropyldimethicone; and silicone elastomers such as the products sold under the name KSG by Shin-Etsu, under the names TREFIL, BY29 or EPSX by Dow Corning or under the name GRANSIL by Grant Industries.

These fatty substances may be chosen in a varied manner by a person skilled in the art in order to prepare a composition having the desired properties, for example consistency or texture properties.

According to a particular embodiment of the invention, the composition according to the invention is a water-in-oil (W/O) or oil-in-water (O/W) emulsion. The proportion of the oily phase of the emulsion may range from 5 to 80 wt %, and preferably from 5 to 50 wt %, relative to the total weight of the composition.

The emulsions generally contain at least one emulsifier chosen from amphoteric, anionic, cationic or non-ionic emulsifiers, used alone or as a mixture, and optionally a co-emulsifier. The emulsifiers are chosen in a suitable manner according to the emulsion to be obtained (W/O or O/W). The emulsifier and the co-emulsifier are generally present in the composition, in an amount ranging from 0.3 to 30 wt %, and preferably from 0.5 to 20 wt % relative to the total weight of the composition.

For W/O emulsions, as emulsifiers, mention may be made, for example, of dimethicone copolyols such as the blend of cyclomethicone and dimethicone copolyol, sold under the name DC 5225 C by Dow Corning, and alkyldimethicone copolyols such as laurylmethicone copolyol sold under the name “Dow Corning 5200 Formulation Aid” by Dow Corning and cetyldimethicone copolyol sold under the name ABIL EM 90® by Goldschmidt.

It is also possible to use, as a surfactant for W/O emulsions, a crosslinked solid organopolysiloxane elastomer comprising at least one oxyalkylene group, such as those obtained according to the procedure in Examples 3, 4 and 8 of document U.S. Pat. No. 5,412,004 and the examples from document U.S. Pat. No. 5,811,487, especially the product from Example 3 (synthetic example) from Patent U.S. Pat. No. 5,412,004 and that sold under the reference KSG 21 by Shin Etsu. Other types of KSGs sold by Shin Etsu may also be used, such as KSG-16.

For O/W emulsions, as emulsifiers, mention may be made, for example, of non-ionic emulsifiers such as oxyalkylenated (more particularly polyoxyethylenated) fatty acid and glycerol esters; oxyalkylenated fatty acid and sorbitan esters; oxyalkylenated (oxyethylenated and/or oxypropylenated) fatty acid esters; oxyalkylenated (oxyethylenated and/or oxypropylenated) fatty alcohol ethers; sugar esters such as sucrose stearate; and mixtures thereof such as the mixture of glyceryl stearate and PEG-40 stearate.

In a known manner, the cosmetic or dermatological composition of the invention may also contain customary adjuvants used in the cosmetic or dermatological field, such as hydrophilic or lipophilic gelling agents, preservatives, solvents, fragrances, fillers, UV screening agents, bactericides, odour absorbers, colouring materials, plant extracts, salts, antioxidants, basic agents, acids, and non-ionic, anionic or cationic surfactants.

The amounts of these various adjuvants are those conventionally used in the field considered, and are for example from 0.01 to 20% of the total weight of the composition. These adjuvants, depending on their nature, may be introduced into the fatty phase, into the aqueous phase and/or into the lipid vesicles.

As fillers that can be used in the composition of the invention, mention may be made, for example, besides pigments, of silica powder, a colloidal amorphous silica; talc; polyamide particles and especially those sold under the name ORGASOL by Atochem; polyethylene powders; microspheres based on acrylic copolymers, such as those made of the ethylene glycol dimethacrylate/lauryl methacrylate copolymer sold by Dow Corning under the name POLYTRAP; expanded powders such as hollow microspheres and especially the microspheres sold under the name EXPANCEL by Kemanord Plast or under the name MICROPEARL F 80 ED by Matsumoto; silicone resin microbeads such as those sold under the name TOSPEARL by Toshiba Silicone; and mixtures thereof. These fillers may be present in amounts ranging from 0 to 20 wt % and preferably from 1 to 10 wt % relative to the total weight of the composition.

As hydrophilic or lipophilic gelling agents, mention may especially be made of CARBOPOL, LUVIGEL, HOSTACERIN AMPS, SIMULGEL, SEPIGEL type acrylamide gelling agents such as SEPIGEL 305® from Seppic, xanthan, guar and cellulose gums, alginates and mixtures thereof. Hectorites may also be mentioned.

As antioxidants, mention may especially be made of polyphenols, tannic acid, epigallocatechins and the natural extracts containing them, anthocyanins, rosemary extracts, olive leaf extracts, green tea, resveratrol and its derivatives, pycnogenol, ergothineine, N-acetylcysteine, biotin, chelating agents, idebenone, plant extracts such as PRONALEN BIOPROTECT™ from Provital, anti-free radical agents such as vitamin E, co-enzyme Q10, bioflavonoids, SOD, phytantriol, lignans, melatonin, pidolates, gluthatione.

According to a preferred embodiment of the invention, the composition used according to the invention contains at least one UV screening agent (or sunscreen) which may be a chemical screening agent or a physical screening agent or a mixture of such screening agents.

By way of illustration and in a non-limiting way, the following families may be mentioned (the names corresponding to the CTFA nomenclature for screening agents):

anthranilates, in particular menthyl anthranilate; benzophenones, in particular benzophenone-1, benzophenone-3, benzophenone-5, benzophenone-6, benzophenone-8, benzophenone-9, benzophenone-12, and preferentially benzophenone-2 (oxybenzone) or benzophenone-4 (UVINUL MS40 available from BASF); benzylidenecamphors, in particular 3-benzylidenecamphor, benzylidenecamphorsulphonic acid, camphor benzalkonium methosulphate, polyacrylamidomethylbenzylidene camphor, terephthalylidenedicamphor-sulphonic acid, and preferentially 4-methylbenzylidene camphor (EUSOLEX 6300 available from Merck); benzimidazols, in particular benzimidazilate (NEO HELIOPAN AP available from Haarmann and Reimer), or phenylbenzimidazolsulphonic acid (EUSOLEX 232 available from Merck); benzotriazols, in particular drometrizole trisiloxane or methylenebis[(benzotriazol-yl)(tetramethylbutyl)phenol] (TINOSORB M available from Ciba); cinnamates, in particular cinoxate, DEA methoxycinnamate, diisopropyl methylcinnamate, glycerylethylhexanoate dimethoxycinnamate, isopropyl methoxycinnamate, isoamyl cinnamate and preferentially ethocrylene (UVINUL N35 available from BASF), octyl methoxycinnamate (PARSOL MCX available from Hoffmann La Roche), or octocrylene (UVINUL 539 available from BASF); dibenzoylmethanes, in particular butyl methoxydibenzoylmethane (PARSOL 1789); imidazolines, in particular ethylhexyl dimethoxybenzylidene dioxoimidazoline; PABAs, in particular ethyldihydroxypropyl PABA, ethylhexyldimethyl PABA, glyceryl PABA, PABA, PEG-25 PABA, and preferentially diethylhexyl butamido triazone (UVASORB HEB available from 3V Sigma), ethylhexyl triazone (UVINUL T150 available from BASF), or ethyl PABA (benzocaine); salicylates, in particular dipropylene glycol salicylate, ethylhexyl salicylate, homosalate, or TEA salicylate; triazines, in particular anisotriazine (TINOSORB S available from Ciba); drometrizole trisiloxane, zinc oxide, titanium dioxide, and coated or uncoated zinc, iron, zirconium or cerium oxides.

The amount of screening agents depend on the final desired use. It may range, for example, from 1 to 20 wt % and better from 2 to 10 wt % relative to the total weight of the composition.

The compositions according to the invention could be applied directly onto the skin or, alternatively, onto cosmetic or dermatological supports of the occlusive or non-occlusive type, intended to be applied to the skin in a localized fashion.

The support may be an “occlusive” support. By way of example, the support is formed from a thermoplastic, chosen from high- and low-density polyethylenes, polypropylenes, polyvinyl chlorides, ethylene/vinyl acetate copolymers, polyesters and polyurethanes, or from a complex of such materials. These materials may also be present in laminated form with at least one metal sheet, such as an aluminium foil.

The support layer may be of any appropriate thickness which will provide the desired support and protective functions. Preferably, the thickness of the support layer is between about 20 μm and about 1.5 mm. Advantageously, the support layer is sufficiently flexible so as to be able to perfectly fit the shape of the skin, and so as not to cause a feeling of discomfort in the user.

Preferably however, the support is “non-occlusive”. In the latter assumption, a support is advantageously used that is formed from a paper, a porous or perforated thermoplastic, a woven fabric, a non-woven fabric or a perforated non-woven fabric.

According to another embodiment of the invention, said compositions according to the invention may be combined with compositions administered orally, containing additional cosmetic active agents having a beneficial effect on the appearance of the skin, such as for example additional active agents intended to combat the signs of skin ageing or additional active agents intended to combat greasy skin.

The additional ingredient used in the composition of the invention may also be a cosmetic or pharmaceutical ingredient and/or active agent, in particular ingredients and/or active agents that are beneficial for the appearance and/or texture of the skin.

According to one particular embodiment of the invention, the composition according to the invention comprises at least one ingredient for solubilizing and/or stabilizing said C-glycoside derivative and, in addition, at least one other additional ingredient and/or active agent chosen from a cosmetic or pharmaceutical ingredient and/or active agent.

C—Additional Cosmetic and Dermatological Active Agents

The additional active agents could especially be chosen from depigmenting agents, antimicrobial agents, antiperspirant agents, metal chelating agents, hydrolysed proteins, antioxidants, vitamins, anti-inflammatory agents, anti-irritant or soothing agents, moisturizing agents, plant extracts, agents improving the barrier function, matifying agents, abrasive fillers or exfoliating agents, desquamating agents, sebum-regulating agents, wound-healing agents, astringent agents, fillers, optical brighteners, fluorescent agents, agents stimulating the synthesis of dermal and/or epidermal macromolecules and/or preventing their degradation, agents stimulating the proliferation of fibroblasts or keratinocytes and/or the differentiation of the keratinocytes, agents favouring the skin barrier function, agents that favour maturing of the horny envelope, NO-synthase inhibitors, peripheral benzodiazepine receptor antagonists, agents increasing the activity of the sebaceous gland, anti-glycation agents, skin-relaxing or dermo-decontracting agents, agents favouring skin microcirculation, agents stimulating cell energy metabolism, tightening agents, slimming or lipolytic agents and mixtures thereof.

As humectant or moisturizing agents, mention may especially be made of glycerol and its derivatives, urea and its derivatives, especially HYDROVANCE® sold by National Starch, lactic acids, hyaluronic acid, AHAs, BHAs, sodium pidolate, xylitol, serine, sodium lactate, ectoine and its derivatives, chitosan and its derivatives, collagen, plankton, an Imperata cylindra extract sold under the name MOIST 24® by Sederma, acrylic acid homopolymers such as LIPIDURE-HM® by NOF Corporation, beta-glucan and in particular sodium carboxymethyl beta-glucan from Mibelle-AG-Biochemistry; a mixture of passion flower, apricot and maize oils, and rice bran sold by Nestle under the name NutraLipids®.

As agents that improve the barrier function, mention may especially be made of ceramides and derivatives, sphingoid-based compounds, glycosphingolipids, phospholipids, cholesterol and its derivatives, phytosterols, essential fatty acids, diacylglycerol, 4-chromanone and chromone derivatives, petroleum jelly, lanolin, shea butter, cocoa butter, lanolin, and PCA salts.

As depigmenting agents, mention may especially be made of vitamin C and its derivatives and especially vitamin CG, vitamin CP and 3-O-ethyl-vitamin C, alpha- and beta-arbutin, lucinol and its derivatives, kojic acid, resorcinol and its derivatives, tranexamic acid and its derivatives, gentisic acid, homogentisate, methyl gentisate or homogentisate, dioic acid, calcium D-pantetheine sulphonate, lipoic acid, ellagic acid, vitamin B3, transexamic acid, lipoic acid, linoleic acid and its derivatives, ceramides and their homologues, derivatives of plants such as camomile, bearberry, the aloe (vera, ferox, bardensis) family, mulberry and skullcap, this list not being exhaustive.

As slimming (lipolytic) agents, mention may especially be made of theophylline and its derivatives, theobromine, acefylline, aminophylline, chloroethyltheophylline, diprofylline, diniprophylline, etamiphylline and its derivatives, etofylline, and proxyphylline; extracts of tea, coffee, guarana, maté, cola (Cola nitida) and especially the dry extract of guarana (Paulina sorbilis) fruit containing 8 to 10% of caffeine; extracts of English ivy (Hedera helix), of arnica (Arnica montana L), of rosemary (Rosmarinus officinalis N), of marigold (Calendula officinalis), of sage (Salvia officinalis L), of ginseng (Panax ginseng), of St John's Wort (Hyperycum perforatum), of butcher's broom (Ruscus aculeatus L), of meadowsweet (Filipendula ulmaria L), of Java tea (Orthosiphon stamincus benth), of birch (Betula alba), of cecropia and argan; ginkgo biloba extracts, extracts of horse tail, escin extracts, cangzhu extracts, extracts of Chrysanthellum indicum, dioscorea extracts rich in diosgenin or pure diosgenin or hecogenin and their derivatives, ballota extracts, extracts of Guioa, Davallia, Terminalia, Barringtonia, Trema, Antirobia and petitgrain bigarade extract.

According to one particular embodiment of the invention, it will be possible to use additional ingredients and/or active agents for the care of greasy skin and/or for combating the signs of skin ageing.

a) Additional Active Agents and/or Ingredients for the Care of Greasy Skin

A composition according to the invention intended for the care of greasy skin, will advantageously comprise an additional ingredient and/or active agent chosen from matifying agents, abrasive fillers or exfoliating agents, desquamating agents, antimicrobial agents, soothing agents, anti-inflammatory agents, sebum-regulating agents, antioxidants, wound-healing agents, astringent agents and mixtures thereof.

According to a particular embodiment of the invention, the composition according to the invention comprises at least one ingredient for solubilizing and/or stabilizing said C-glycoside derivative and, in addition, at least one other additional ingredient and/or active agent for the care of greasy skin.

Examples of such additional ingredients and/or active agents are described below.

Matifying Agents

The term “matifying agent” is understood to mean agents intended to make the skin visibly more matt, less shiny.

The matifying effect of the agent and/or of the composition containing it may especially be evaluated using a gonioreflectometer, by measuring the ratio R between the specular reflection and the diffuse reflection. An R value less than or equal to 2 generally indicates a matifying effect.

The matifying agent may especially be chosen from a rice starch, a maize starch, kaolinite, silicas, talc, pumpkin seed extract, cellulose microbeads, plant fibres, synthetic fibres, in particular polyamide fibres, expanded acrylic copolymer microspheres, polyamide powders, silica powders, polytetrafluoroethylene powders, silicone resin powders, acrylic copolymer powders, wax powders, polyethylene powders, crosslinked organopolysiloxane elastomer powders coated with silicone resin, composite talc/titanium dioxide/alumina/silica powders, amorphous mixed silicate powders, acrylic polymer powders, silicate particles and especially mixed silicate particles, and mixtures thereof.

As examples of matifying agents, mention may especially be made of:
rice or maize starch, in particular an aluminium starch octenylsuccinate sold under the name DRY-FLO® by National Starch;
kaolinite;
silicas;
talc;
a pumpkin seed extract as sold under the name CURBILENE® by Indena;
cellulose microbeads as described in L'Oréal Patent Application EP 1 562 562;
fibres, such as silk fibres, cotton fibres, wool fibres, flax fibres, fibres of cellulose extracted especially from wood, vegetables or algae, polyamide (Nylon®) fibres, modified cellulose fibres, poly(p-phenylene terephthalamide) fibres, acrylic fibres, polyolefin fibres, glass fibres, silica fibres, aramid fibres, carbon fibres, Teflon® fibres, insoluble collagen fibres, polyester fibres, polyvinyl chloride or polyvinylidene chloride fibres, polyvinyl alcohol fibres, polyacrylonitrile fibres, chitosan fibres, polyurethane fibres, polyethylene phthalate fibres, fibres formed from a blend of polymers, resorbable synthetic fibres, and mixtures thereof described in L'Oréal Patent Application EP 1 151 742;
expanded acrylic copolymer microspheres such as those sold by Expancel under the name EXPANCEL 551®;
fillers having an optical effect as described in Patent Application FR 2 869 796, in particular:
   polyamide (Nylon®) powders, such as for example ORGASOL-type nylon-12 particles from Atofina, having an average size of 10 microns and a refractive index of 1.54;
   silica powders, such as for example the silica beads SB150 from Miyoshi, having an average size of 5 microns and a refractive index of 1.45;
   polytetrafluoroethylene powders, such as the CERIDUST 9205F PTFEs from Clariant, having an average size of 8 microns and a refractive index of 1.36;
   silicone resin powders such as the TOSPEARL 145A silicone resin from GE Silicone, having an average size of 4.5 microns and a refractive index of 1.41;
   acrylic copolymer powders, especially polymethyl(meth)acrylate powders, such as the JURYMER MBI PMMA particles from Nihon Junyoki, having an average size of 8 microns and a refractive index of 1.49, or the MICROPEARL M100® and F 80 ED® particles from Matsumoto Yushi-Seiyaku;
   wax powders such as MICROEASE 114S paraffin wax particles from MicroPowders, having an average size of 7 microns and a refractive index of 1.54;
   polyethylene powders, especially comprising at least one ethylene/acrylic acid copolymer, and in particular formed from ethylene/acrylic acid copolymers such as FLOBEADS EA 209 particles from Sumitomo (having an average size of 10 microns and a refractive index of 1.48);
   crosslinked organopolysiloxane elastomer powders coated in silicone resin, especially silsesquioxane resin, as described, for example, in Patent U.S. Pat. No. 5,538,793. Such elastomer powders are sold under the names KSP-100, KSP-101, KSP-102, KSP-103, KSP-104 and KSP-105 by Shin-Etsu; and
   composite talc/titanium dioxide/alumina/silica powders such as those sold under the name COVERLEAF AR-80 by Catalyst & Chemicals,

and mixtures thereof,
compounds that absorb and/or adsorb sebum as described in the same Patent Application FR 2 869 796. Mention may especially be made of:
   silica powders, such as for example porous silica microspheres sold under the name SILICA BEADS SB-700 sold by Myoshi, SUNSPHERE® H51, SUNSPHERE® H33 and SUNSPHERE® H53 sold by Asahi Glass; amorphous silica microspheres coated with polydimethylsiloxane sold under the name SA SUNSPHERE® H-33 and SA SUNSPHERE® H53 sold by Asahi Glass;
   amorphous mixed silicate powders, especially of aluminium and magnesium, such as for example the one sold under the name NEUSILIN UFL2 by Sumitomo;
   polyamide (nylon®) powders, such as for example ORGASOL® 4000 sold by Atochem;
   acrylic polymer powders, especially polymethyl methacrylate powders, such as for example COVABEAD® LH85 sold by Wackherr; polymethyl methacrylate/ethylene glycol dimethacrylate powders, such as for example DOW CORNING 5640 MICROSPONGE® SKIN OIL ADSORBER sold by Dow Corning, or GANZPEARL® GMP-0820 sold by Ganz Chemical; polyallyl methacrylate/ethylene glycol dimethacrylate powders, such as for example POLY-PORE® L200 or POLY-PORE® E200 sold by AMCOL; ethylene glycol dimethacrylate/lauryl methacrylate copolymer powders, such as for example POLYTRAP® 6603 sold by Dow Corning;
   silicate particles, such as alumina silicate; and
   mixed silicate particles, such as:
      magnesium-aluminium silicate particles, such as saponite or magnesium-aluminium silicate hydrated with a sodium sulphate sold under the trade name SUMECTON® by Kunimine; and
      the complex of magnesium silicate, hydroxyethyl cellulose, black cumin oil, pumpkin oil and phospholipids, or MATIPURE® from Lucas Meyer,

and mixtures thereof.

As preferred matifying agents, it will be possible to use, according to the invention, a pumpkin seed extract, a rice or maize starch, kaolinite, silicas, talc, polyamide powders, polyethylene powders, acrylic copolymer powders, expanded acrylic copolymer microspheres, silicon resin microbeads, mixed silicate particles and mixtures thereof.

Abrasive Fillers or Exfoliating Agents

As exfoliating agents that can be used in the rinse-off compositions according to the invention, mention may be made, for example, of exfoliating or scrubbing particles of mineral, plant or organic origin. Thus, it is possible to use, for example, polyethylene beads or powder, nylon powder, polyvinyl chloride powder, pumice, ground products of apricot kernels or of nut shells, sawdust, glass beads, alumina, and mixtures thereof.

Mention may also be made of EXFOGREEN® from Solabia (bamboo extract), extracts of strawberry achenes (strawberry achenes from Greentech), peach kernel powder, apricot kernel powder, and finally in the field of plant powders having an abrasive effect, mention is made of cranberry seed powder.

As abrasive fillers or exfoliating agents that are preferred according to the invention, mention will be made of peach kernel powder, apricot kernel powder, cranberry seed powder, extracts of strawberry achenes and bamboo extracts.

The expression “additional active agent for the care of greasy skin” is understood to mean, within the scope of the present invention, a compound which has by itself, that is to say not requiring the intervention of an external agent to activate it, a biological activity which may be, in particular:

a desquamating activity (which allows opening of the comedones); and/or

an antimicrobial activity (especially on P. acnes); and/or

a soothing or anti-inflammatory activity; and/or

a sebum-regulating activity; and/or

an antioxidant activity (which prevents the oxidation of squalene and the formation of comedones);

a wound-healing activity; or

an astringent activity.

As examples of active agents combined with the C-glycoside derivatives that can be used in the compositions of the invention, mention may therefore be made of desquamating agents, antimicrobial agents, soothing agents, anti-inflammatory agents, sebum-regulating agents, antioxidant agents and mixtures thereof.

Preferably, the composition according to the invention will comprise, as additional active agent, at least one sebum-regulating agent.

According to one particular embodiment, the composition could comprise, in addition, optionally an antimicrobial agent.

More preferably, it could comprise, in addition, a desquamating agent.

And to further increase the effectiveness and/or tolerance of said compositions, it will be possible to add, in addition, soothing or anti-inflammatory agents, antioxidant agents, wound-healing agents, astringent agents, and mixtures thereof.

As examples of compounds, for each class, mention may especially be made of:

Sebum-Regulating or Antiseborrheic Agents

The term “sebum-regulating or antiseborrheic” agents is understood to mean especially agents capable of regulating the activity of the sebaceous glands.

Mention may especially be made of:

retinoic acid, benzoyl peroxide, sulphur, vitamin B6 (or pyridoxine), selenium chloride, and sea fennel;

mixtures of cinnamon, tea and octanoyl glycine extract such as SEPICONTROL A5 TEA from Seppic;

the mixture of capryloyl glycine, sarcosine and cinnamomum zeylanicum extract sold especially by Seppic under the trade name SEPICONTROL A5®;

zinc salts such as zinc gluconate, zinc pyrrolidone carboxylate (or zinc pidolate), zinc lactate, zinc aspartate, zinc carboxylate, zinc salicylate and zinc cysteate;

copper salts, in particular copper pidolate;

plant extracts of the species Arnica montana, Cinchona succirubra, Eugenia caryophyllata, Humulus lupulus, Hypericum perforatum, Mentha piperita, Rosmarinus officinalis, Salvia officinalis and Thymus vulgaris, all sold, for example, by Maruzen;

extracts of meadowsweet (Spiraea ulamaria) such as that sold under the name SEBONORMINE® by Silab;

extracts of the Laminaria saccharina algae such as that sold under the name PHLOROGINE® by Biotechmarine;

sugar cane extracts, such as that sold under the name POLICASONOL® by Sabinsa;

mixtures of extracts of burnet (Sanguisorba officinalis/Poterium officinale) roots, ginger (Zingiber officinalis) rhizomes and cinnamon (Cinnamomum cassia) bark such as that sold under the name SEBUSTOP® by Solabia;

linseed extracts such as that sold under the name LINUMINE® by Lucas Meyer;

Phellodendron extracts such as those sold under the name PHELLODENDRON EXTRACT BG by Maruzen or OUBAKU LIQUID B by Ichimaru Pharcos;

mixtures of argan oil, saw palmetto extract and sesame seed extract such as that sold under the name REGU SEB® by Pentapharm;

mixtures of extracts of rosebay willow herb, Terminalia chebula, nasturtium and bioavailable zinc (microalgae) such as that sold under the name SEBORILYS® by Greentech;

extracts of Pygeum africanum such as that sold under the name PYGEUM AFRICANUM STEROLIC LIPID EXTRACT by Euromed;

extracts of saw palmetto such as those sold under the name VIAPURE SABAL by Actives International, or those sold by Euromed;

mixtures of extracts of plantain, Berberis aquifolium and sodium salicylate such as that sold under the name SEBOCLEAR® by Rahn;

clove extract such as that sold under the name CLOVE EXTRACT POWDER by Maruzen;

argan oil such as that sold under the name LIPOFRUCTYL® by Laboratories Sérobiologiques;

lactic protein filtrates such as that sold under the name NORMASEB® by Sederma;

extracts of Laminaria algae, such as that sold under the name LAMINARGHANE® by Biotechmarine;

oligosaccharides of Laminaria digitata algae such as that sold under the name PHYCOSACCHARIDE AC by Codif;

sulphonated shale oil, such as that sold under the name ICHTYOL PALE by Ichthyol;

meadowsweet (Spiraea ulmaria) extracts, such as that sold under the name CYTOBIOL ULMAIRE by Libiol;

sebacic acid, especially sold in the form of a sodium polyacrylate gel under the name SEBOSOFT by Sederma;

glucomannans extracted from konjac tuber and modified by alkylsulphonate chains, such as that sold under the name BIOPOL BETA by Arch Chemical;

extracts of Sophora angustifolia, such as those sold under the name SOPHORA POWDER or SOPHORA EXTRACT by Bioland;

extracts of Cinchona succirubra bark such as that sold under the name RED BARK HS by Alban Muller;

extracts of Quillaja saponaria such as that sold under the name PANAMA WOOD HS by Alban Muller;

glycine grafted onto an undecylene chain, such as that sold under the name LIPACIDE UG OR by Seppic;

mixture of oleanolic acid and nordihydroguaiaretic, such as that sold in the form of a gel under the name AC.NET by Sederma;

phthalimidoperoxyhexanoic acid;

trialkyl (C₁₂-C₁₃) citrate sold under the name COSMACOL® ECI by Sasol; trialkyl (C₁₄-C₁₅) citrate sold under the name COSMACOL® ECL by Sasol;

10-hydroxydecanoic acid, and especially mixtures of 10-hydroxydecanoic acid, sebacic acid and 1,10-decanediol such as that sold under the name ACNACIDOL® BG by Vincience;

specific PPAR-γ activators such as those described in Application WO 2005/053632;

extracts of plants of the Silybum genus;

sapogenins or plant extracts containing them, in particular extracts of Dioscorea rich in diosgenin; and

extracts of Eugenia caryophyllata containing eugenol and eugenyl glycoside, and mixtures thereof.

As preferred sebum-regulating agents that can be used according to the invention, mention will be made of:

sea fennel;

mixtures of cinnamon, tea and octanoyl glycine extract such as SEPICONTROL A5 TEA from Seppic;

the mixture of capryloyl glycine, sarcosine and Cinnamomum zelanicum extract especially sold by Seppic under the trade name SEPICONTROL A5®;

zinc salts such as zinc gluconate, zinc pyrrolidone carboxylate (or zinc pidolate), zinc lactate, zinc aspartate, zinc carboxylate, zinc salicylate and zinc cysteate;

copper salts, in particular copper pidolate;

extracts of meadowsweet (Spiraea ulamaria) such as that sold under the name SEBONORMINE® by Silab;

extracts of Laminaria saccharina algae such as that sold under the name PHLOROGINE® by Biotechmarine;

mixtures of extracts of burnet (Sanguisorba officinalis/Poterium officinale) roots, ginger (Zingiber Officinalis) rhizomes and cinnamon (Cinnamomum cassia) bark such as that sold under the name SEBUSTOP® by Solabia;

sapogenins or plant extracts containing them, in particular extracts of Dioscorea rich in diosgenin,

and mixtures thereof.

Mention may also be made of antiperspirants, such as: aluminium and/or zirconium salts; complexes of zirconium chlorohydrate and aluminium chlorohydrate with an amino acid such as those described in Patent U.S. Pat. No. 3,792,068 commonly known by the name “ZAG complexes”. Such complexes are generally known by the name ZAG (when the amino acid is glycine). ZAG complexes usually have an Al/Zr ratio ranging from around 1.67 to 12.5 and a metal/Cl ratio ranging from around 0.73 to 1.93. Among these products, mention may be made of aluminium zirconium octachlorohydrex GLY, aluminium zirconium pentachlorohydrex GLY, aluminium zirconium tetrachlorohydrate GLY and aluminium zirconium trichlorohydrate GLY.

Among the aluminium salts, mention may be made of aluminium chlorohydrate, aluminium chlorohydrex, aluminium chlorohydrex PEG, aluminium chlorohydrex PG, aluminium dichlorohydrate, aluminium dichlorohydrex PEG, aluminium dichlorohydrex PG, aluminium sesquichlorohydrate, aluminium sesquichlorohydrex PEG, aluminium sesquichlorohydrex PG, alum salts, aluminium sulphate, aluminium zirconium octachlorohydrate, aluminium zirconium pentachlorohydrate, aluminium zirconium tetrachlorohydrate, aluminium zirconium trichlorohydrate and more particularly aluminium chlorohydrate sold by Reheis under the name MICRODRY ALUMINUM CHLOROHYDRATE or by Guilini Chemie under the name ALOXICOLL PF 40. Aluminium zirconium salts are for example the one sold by Reheis under the name REACH AZP-908-SUF, “activated” aluminium salts, for example the one sold by Reheis under the name REACH 103 or by Westwood under the name WESTCHLOR 200.

Among the other deodorant active agents, mention may also be made of zinc salts such as zinc salicylate, zinc sulphate, zinc chloride, zinc lactate, and zinc phenolsulphonate; chlorhexidine and its salts; diglycerol monocaprate, diglycerol monolaurate, glycerol monolaurate; and polyhexamethylene biguanide salts.

Antimicrobial Agents

The term “antimicrobial agents” is understood to mean agents having effects on the specific flora of greasy skin, such as for example P. acnes.

These effects may be either bactericidal or resistant to bacterial adhesion (prevent and/or reduce the adhesion of microorganisms), or acting on the biofilm of the bacteria to prevent them from multiplying.

Mention may especially be made of the active agents and preservatives having antimicrobial activity cited in Application DE 10324567, incorporated into the present invention by reference.

Mention may also be made of: a hop cone extract (HOP CO2-TO EXTRACT from Flavex), an extract of St John's Wort (ST JOHN'S WORT CO2-TO EXTRACT from Flavex), asiatic acid, extracts of Scutellaria baicolensis roots such as in BMB-CF from Naturogin, piroctone olamine, citrollic acid, sperillic acid, ethylhexyl glycerine (SENSIVA from Shulke), gluceryl caprylate/caprate (CAPMUL from Abitec), calcium sodium phosphosilicate such as BIOACTIVE GLASS POWDER from Schott, ACTYSSE PREMIER BG from Schott, silicon oxides from Ciba, METASHINES (derivatives of silver), bearberry extracts such as GATULINE EQUALIZING from Gattefosse, 10-hydroxy-2-decanoic acid such as ACNACIDOL P from Vincience, sodium ursolate, azelaic acid, diiodomethyl P-tolylsulphone or AMICAL FLOWABLE from Angus, MALACHITE from Maprecos, ZINCARE from Elementis GmbH, ARLATONE DIOIC from Unichema, phthalimidoperoxyhexanoic acid or EURECO HC from Chemron Corporation; ellagic acid; 2,4,4′-trichloro-2′-hydroxydiphenyl ether (or Triclosan), 1-(3′-4′-dichlorophenyl)-3-(4′-chlorophenyl)urea (or Triclocarban), 3,4,4′-trichlorocarbanilide, 3′,4′,5′-trichlorosalicylanilide, phenoxyethanol, phenoxypropanol, phenoxyisopropanol, hexamidine isethionate, metronidazole and its salts, micronazole and its salts, itraconazole, terconazole, econazole, ketoconazole, saperconazole, fluconazole, clotrimazole, butoconazole, oxiconazole, sulphaconazole, sulconazole, terbinafine, ciclopirox, ciclopiroxolamine, undecylenic acid and its salts, benzoyl peroxide, 3-hydroxybenzoic acid, 4-hydroxybenzoic acid, phytic acid, N-acetyl-L-cysteine, lipoic acid, azelaic acid and its salts, arachidonic acid, resorcinol, 3,4,4′-trichlorocarbanalide, octopirox or piroctone olamine, octoxyglycerine or octoglycerine, octanoyl glycine (LIPACID C8G® from Seppic), caprylyl glycol, 10-hydroxy-2-decanoic acid, dichlorophenyl imidazole dioxolane and its derivatives described in Patent Application WO 9318743, zinc derivatives and in particular zinc pidolate (ZINCIDONE® from Solabia), copper derivatives and in particular copper pidolate (CUIVRIDONE® from Solabia), salicylic acid and its derivatives, iodopropynyl butylcarbamate, 3,7,11-trimethyl-dodeca-2,5,10-trienol or farnesol, phytosphingosines; SEPICONTROL® from Seppic, an argan extract such as ARGAPURE LS9710®, SEBOSOFT® from Sederma, quaternary ammonium salts such as cetyltrimethylammonium salts, cetylpyridinium salts, ethanol, etc. and mixtures thereof.

As agents that prevent and/or reduce the adhesion of microorganisms, mention may especially be made of phytantriol and its derivatives as described in Patent Application EP 1 529 523, plant oils such as wheat germ oil, calendula oil, caster oil, olive oil, avocado oil, sweet almond oil, peanut oil, jojoba oil, sesame oil, apricot kernel oil, sunflower oil, and macadamia oil, described in Patent EP 1 133 979, or else other fatty substances such as disodium cocoamphodiacetate, oxyethylenated (7 EO) glyceryl cocoate, poloxamers, hexadecenyl succinate 18, octoxyglyceryl palmitate, octoxyglyceryl behenate, dioctyl adipate, PPG-15 stearyl ether, branched C₁₂-C₁₃ dialcohol tartrate described in Patent EP 1 129 694.

In particular, as regards the propagation of P. acnes, mention may be made of pentylene glycol, nylon-6,6 (polyamide PA-6,6 fibres), rice bran oil, CELVOL 540 PV alcohol (polyvinyl alcohol 72962), AKOREX L from Karlshamns, and fructose derivatives.

Mention may also be made of certain surfactants having an antimicrobial effect such as sodium cocoamphoacetate or disodium cocoamphodiacetate such as MIRANOL C2M CONC NP, betaines such as GENAGEN KB cocoyl betaine from Clariant, sodium lauryl ether sulphate such as EMAL 270 D from Kao, decyl glucoside such as PLANTACARE 2000 UP, branched C₁₂-C₁₃ dialcohol malate such as COSMACOL EMI, propylene glycol monoesters such as propylene glycol monolaurate, monocaprylate or monocaprate, sodium lauroyl oat amino acid such as PROTEOL OAT, lauryl dimethylamine betaine such as EMPIGEN BB/LS and also polyquaternary ammonium compounds such as QUATERNIUM-24 or BARDAC 2050 from Lonza and those described in L'Oréal Patent FR 0 108 283.

As preferred antimicrobial agents, an agent will be used in the compositions of the invention chosen from caprylyl glycol, zinc derivatives including zinc pidolate (ZINCIDONE® from Solabia), copper derivatives including copper pidolate (CUIVRIDONE® from Solabia) octoglycerine or octoxyglycerine, 10-hydroxy-2-decanoic acid, and mixtures thereof.

Desquamating Agents

The term “desquamating agent” is understood to mean any compound capable of acting:

either directly on desquamation by favouring exfoliation, such as β-hydroxy acids, in particular salicylic acid and its derivatives (including 5-(n-octanoyl)salicylic acid); α-hydroxy acids, such as glycolic acid, citric acid, lactic acid, tartaric acid, malic acid or mandelic acid; urea; gentisic acid and its derivatives; oligofucoses; cinnamic acid; Saphora japonica extract; resveratrol and some jasmonic acid derivatives;

or on the enzymes involved in desquamating or degrading the corneodesmosomes, glycosidases, stratum corneum chymotryptic enzyme (SCCE) or even other proteases (trypsin, chymotrypsin-like). Mention may be made of aminosulphonic compounds and in particular N-(2-hydroxyethyl)piperazine-N-2-ethanesulphonic acid (HEPES); 2-oxothiazolidine-4-carboxylic acid (procysteine) derivatives; derivatives of glycine-type α-amino acids (as described in EP 0 852 949, and also sodium methylglycinediacetate sold by BASF under the trade name TRILON M); honey; sugar derivatives such as O-octanoyl-6-D-maltose and N-acetylglucosamine.

As other desquamating agents that can be used in the composition according to the invention, mention may be made of oligofructoses, EDTA and its derivatives, Laminaria extracts, O-linoleyl-6-D-glucose, (3-hydroxy-2-pentylcyclopentyl)acetic acid, glycerol trilactate, O-octanyl-6′-D-maltose, S-carboxymethyl cysteine, silicon-containing salicylate derivatives as in Patent EP 0 796 861, oligofucase as in Patent EP 0 218 200, 5-acylsalicylic acid salts, active agents having effects on transglutaminase as in Patent EP 0 899 330, jasmonic acid and derivatives as in Patent Applications EP 1 333 022 and EP 1 333 021.

EXFOLACTIVE® from Silab (extract of Opuntia ficus-indica flower), SOYPON O® from Kawaken Fine Chemicals (sodium cocoyl sarcosinate).

As preferred desquamating agents, mention may be made of β-hydroxy acids, such as 5-(n-octanoyl)salicylic acid; urea; glycolic acid, citric acid, lactic acid, tartaric acid, malic acid or mandelic acid; N-(2-hydroxyethyl)piperazine-N-2-ethanesulphonic acid (HEPES); Saphora japonica extract; honey; N-acetylglucosamine; sodium methylglycinediacetate, and mixtures thereof.

Even more preferentially, a desquamating agent will be used in the compositions of the invention chosen from 5-(n-octanoyl)salicylic acid; urea; N-(2-hydroxy-ethyl)piperazine-N-2-ethanesulphonic acid (HEPES); Saphora japonica extract; honey; N-acetylglucosamine; sodium methylglycinediacetate, and mixtures thereof.

Soothing or Anti-Irritant Agents

Mention may especially be made of the soothing or anti-irritant agents cited in Applications WO 2004/105736 and DE 10324567, incorporated into the present invention by reference.

As particular soothing agents that can be used in the composition according to the invention, mention may be made of: procyannidolic oligomers, vitamins E, C, B5, B3, dextransulphate, caffeine and its derivatives, pentacyclic triterpenes and the plant extracts containing them, β-glycyrrhetinic acid and its salts or derivatives (stearyl glycyrrhetate, 3-stearoyloxyglycyrrhetinic acid, glycyrrhetinic acid monoglucuronide) and also the plants containing them (e.g. Glycyrrhiza glabra), oleanolic acid and its salts, ursolic acid and its salts, boswellic acid and its salts, betulinic acid and its salts, an extract of Paeonia suffruticosa and/or lactiflora, zinc salicylate, phycosaccharides from Codif, an extract of Laminaria saccharina, extracts of Centella asiatica, canola oil, bisabolol, the phosphoric diester of vitamin E and C such as SEPIVITAL EPC® from Seppic, camomile extracts, allantoin, unsaturated omega-3 oils such as musk rose, blackcurrant, ecchium and fish oils, calophyllum oil, plankton extracts, capryloyl glycine, SEPPICALM VG® (Nymphea alba and sodium palmitoylproline) from Seppic, a Pygeum extract, an extract of Boswellia serrata, an extract of Centipeda cunninghamii, an extract of Helianthus annus in particular HELIOXINE from Silab, an extract of Linum usitatissimum such as SENSILINE from Silab, tocotrienols, extracts of Cola nitida, piperonal, a clove extract, an extract of Epilobium angustifolium, aloe vera, an extract of Bacopa moniera, phytosterols, cornflower water, rose water, dextran such as MODULENE® from Vincience, a mint extract, in particular mint leaves such as CALMISKIN® from Silab, anise derivatives, filamentous bacteria such as Vitreoscilla filiformis as described in Patent EP 761 204, a rose extract such as HERBASOL rose extract, STIMU-TEX AS from Pentapharm, alkaline-earth-metal salts especially strontium, niacinamide, and mixtures thereof.

As preferred soothing agents, an agent will be used chosen from a rose extract, a clove extract, dextran such as MODULENE® from Vincience, a mint extract such as CALMISKIN® from Silab, a mixture of a Nymphea alba extract and sodium palmitoylproline such as SEPPICALM VG® from Seppic, anise derivatives, an extract of Paeonia suffruticosa and/or lactiflora, and mixtures thereof.

Anti-Inflammatory Agents

Mention may especially be made of the anti-inflammatory agents cited in Applications WO 2004/105736 and DE 10324567, incorporated in the present invention by reference.

As particular anti-inflammatory agents that can be used according to the invention, mention may be made of cortisone, hydrocortisone, indomethacin, betamethasone, azelaic acid, acetaminophen, diclofenac, clobetasol propionate and mixtures thereof.

As preferred anti-inflammatory agent, mention will be made of azelaic acid.

Antioxidants

This is understood to mean agents having an antioxidant activity (which prevent the oxidation of squalene and the formation of comedones).

Mention may especially be made of tocopherol and its esters, in particular tocopherol acetate; BHT and BHA.

Mention may also be made of polyphenols, tannic acid, epigallocathechins and the natural extracts containing them, anthocyanins, rosemary extracts, olive leaf extracts, green tea, resveratrol and its derivatives, pycnogenol, ergothineine, N-acetylcysteine, biotin, chelating agents, idebenone, plant extracts such as PRONALEN BIOPROTECT™ from Provital, anti-free radical agents such as vitamin E, co-enzyme Q10, bioflavonoids, SODs, phytantriol, lignans, melatonin, pidolates, and glutathione.

Wound-Healing Agents

As examples of wound-healing agents, mention may especially be made of: allantoin, urea, wheat germ oil, certain amino acids such as hydroxyproline, arginine, serine, glutamic acid and also white lily extracts (e.g. PHYTELENE LYS 37EG 16295 from Indena), a yeast extract such as the wound healer LS 7225B from LS (Cognis), tamanu oil, extract of Saccharomyces cerevisiae or BIODYNES TRF from Arch Chemical, oat extracts, chitosan and derivatives, carrot extracts, artemia extract or GP4G from Vincience, sodium acexamate, lavandin extracts, honey or propolis extracts, ximenynic acid and its salts such as Indena ximenynic acid, Rosa rugosa oil, SOUCI AMI LIPOSOLIBLE from Alban Muller, horsetail extracts, HERBASOL CITRON from Cosmetochem, helichrysum extracts, β-glucan and derivatives, shea butter and its purified fractions, modified exopolysaccharides and alkyl-sulphonated polyaminosaccharides.

As wound-healing agents that are preferred according to the invention, tamanu oil, sodium acexamate, honey extracts, horsetail extracts, helichrysum extracts, and mixtures thereof will be used.

Astringent Agents

The term “astringent agents” is understood according to the invention to mean agents that make it possible to combat dilation of the sebaceous follicles.

As astringent agents that can be used in the composition according to the invention, mention may be made of LARICYL LS8865® from Cognis, PHYTOFIRM LS9120® from Cognis, TANLEX VE/VB® from Ichimaru Pharcos, laponite, aluminium salts, Centella extracts (e.g. PLANTACTIV CENTELLA from Cognis), VARISOFT 432 CG® from Degussa, horse chestnut extracts, extracts of mallow and of Hammamelis, ALMONDERMIN LS 3380® from Cognis, burdock extracts, EXTRAPONE 9 SPECIA®1 from Symrise, skullcap extracts, Ulmaire extracts (e.g. CYTOBIOL ULMAIRE from Libiol), HERB EXTRACT B1348® from Bell Flavors & Fragrances, extracts of acacia, elm, white willow, cinnamon, birch and meadowsweet, panama wood sapogenins, zinc phenolsulphonate from Interchemical, extracts of gentian, cucumber and walnut, and the EPILAMI mixture from Alban Muller.

As astringent agents that are preferred according to the invention, skullcap extracts, Ulmaire extracts, meadowsweet extracts, gentian extracts, burdock extracts and mixtures thereof will be used.

b) Additional Active Agents and/or Ingredients for Combating the Signs of Skin Ageing

A cosmetic anti-ageing composition according to the invention will advantageously comprise, in combination with said C-glycoside derivative, an additional ingredient and/or active agent chosen from: fillers, optical brighteners, fluorescent agents, agents stimulating the synthesis of dermal and/or epidermal macromolecules and/or preventing their degradation, agents stimulating the proliferation of fibroblasts or keratinocytes and/or the differentiation of their keratinocytes, agents favouring the skin barrier function, agents that favour maturing of the horny envelope, NO-synthase inhibitors, peripheral benzodiazepine receptor antagonists, agents increasing the activity of the sebaceous gland, anti-glycation agents, skin-relaxing or dermo-decontracting agents, agents favouring skin microcirculation, agents stimulating cell energy metabolism, tightening agents, and mixtures thereof.

According to one particular embodiment, said composition comprises, in combination with said C-glycoside derivative, at least one additional ingredient favouring the solubilization and/or stabilization of said derivative and at least one additional ingredient and/or active agent chosen from: fillers, optical brighteners, fluorescent agents, agents stimulating the synthesis of dermal and/or epidermal macromolecules and/or preventing their degradation, agents stimulating the proliferation of fibroblasts or keratinocytes and/or the differentiation of their keratinocytes, agents favouring the skin barrier function, agents that favour maturing of the horny envelope, NO-synthase inhibitors, peripheral benzodiazepine receptor antagonists, agents increasing the activity of the sebaceous gland, anti-glycation agents, skin-relaxing or dermo-decontracting agents, agents favouring skin microcirculation, agents stimulating cell energy metabolism, tightening agents, and mixtures thereof.

Examples of such additional ingredients and/or active agents are described below.

I. Concealing Agents, in Particular Fillers

The term “concealing agents” is understood to mean fillers, optical brighteners, fluorescent agents and mixtures thereof.

The term “filler” is understood to mean any material capable of modifying irregularities in the skin microrelief, in particular wrinkles and fine lines, via its intrinsic physical properties and capable of masking them. They are also referred to as fillers having a blurring effect or soft-focus fillers.

As examples of fillers, mention may be made of:
porous silica microparticles such as for example SILICA BEADS SB 150 and SB 700 from Myochi, having an average size of 5 μm and SUNSPHERES SERIES H from Asahi Glass such as H33 and H51, having a size respectively of 3.5 and 5 μm;
hollow hemispherical silicone particles such as the NLK series including NLK 500, NLK 506 and NLK 510 and NLK 523 from Takemoto Oil and Fat. These particles have the CTFA name: methylsilanol/silicate crosspolymer, in particular portions of hollow spheres in the shape of bowls are used. These can be obtained as described in Application JP-2003 128788. Portions of hollow spheres having a horseshoe shape are also described in Application JP-A-2000 191789. As concave particles of portions of spheres that can be used according to the invention, mention can be made of:
silicone resin powders, such as for example the silicone resin TOSPEARL 145 A from GE Silicone, having an average size of 4.5 μm;
acrylic copolymer powders, especially polymethyl(meth)acrylate powders such as for example JURIMER MBI PMMA particles from Nihon Junyoki, having an average size of 8 μm, hollow PMMA spheres sold under the name COVABEAD LH 85 by Wackherr and expanded vinylidene/acrylonitrile/methylene methacrylates microspheres sold under the name EXPANCEL;
wax powders such as MICROASE 114S paraffin wax particles from MicroPowders, having an, average size of 7 μm;
Polyethylene powders, especially comprising at least one ethylene/acrylic acid copolymer, such as for example FLOBEADS EA 209 E from Sumimoto, having an average size of 10 μm;
crosslinked organopolysiloxane elastomer powders coated with silicone resin, especially silsesquioxane powders, sold under the name KSP 100, KSP 101, KSP 102, KSP 103, KSP 104 and KSP 105 by Shin-Etsu;
composite talc/titanium dioxide/alumina/silica powders such as for example COVERLEAF AR 80 from Catalyst & Chemical;
mention may also be made of: talc, mica, kaolin, lauryl glycine, starch powders crosslinked with octeanyl succinate anhydride, boron nitride, polytetrafluoroethylene powders, precipitated calcium carbonate, magnesium hydrogencarbonate carbonate, barium sulphate, hydroxyapatite, calcium silicate, cerium dioxide and glass or ceramic microcapsules; and
fibres, such as silk fibres, cotton fibres, wool fibres, flax fibres, cellulose fibres extracted in particular from wood, vegetables or algae, polyamide (Nylon®) fibres, modified cellulose fibres, poly(p-phenylene terephthalamide) fibres, acrylic fibres, polyolefin fibres, glass fibres, silica fibres, aramid fibres, carbon fibres, Teflon® fibres, insoluble collagen fibres, polyester fibres, polyvinyl chloride fibres or polyvinylidene chloride fibres, polyvinyl alcohol fibres, polyacrylonitrile fibres, chitosan fibres, polyurethane fibres, polyethylene phthalate fibres, fibres formed from a blend of polymers, resorbable synthetic fibres, and mixtures thereof described in L'Oréal Patent Application EP 1 151 742.

The fillers are especially chosen from porous silica microparticles, hollow hemispherical silicone particles, silicone resin powders, acrylic copolymer powders, polyethylene powders, crosslinked organopolysiloxane elastomer powders coated with silicone resin, composite talc/titanium dioxide/alumina/silica powders, precipitated calcium carbonate, magnesium hydrogencarbonate carbonate, barium sulphate, hydroxyapatite, calcium silicate, cerium dioxide and glass or ceramic microcapsules, silk fibres, cotton fibres, and mixtures thereof.

One of the preferred fillers according to the invention is hydroxyapatite.

The concentration of these fillers is generally between 0.1 and 40 wt % relative to the total weight of the composition, preferably from 3.5 to 40 wt %, and even more preferentially from 5 to 15 wt %, relative to the total weight of the composition.

The term “soft-focus filler” is understood to mean a filler which also gives transparency to the skin tone and a blurred effect. Preferably, the soft-focus fillers have an average particle size less than or equal to 15 microns. These particles may be of any shape and in particular may be spherical or non-spherical. More preferably, these fillers are non-spherical.

The soft-focus fillers may be chosen from silica and silicate powders, especially alumina powders, polymethyl methacrylate (PMMA) type powders, talc, silica/TiO₂ or silica/zinc oxide composites, polyethylene powders, starch powders, polyamide powders, styrene/acrylic copolymer powders, silicone elastomers, and mixtures thereof.

In particular, mention may be made of talc having a number-average size less than or equal to 3 microns, for example talc having a number-average size of 1.8 microns and especially that sold under the trade name TALC P3® from Nippon Talc, nylon-12 powder, especially that sold under the name ORGASOL 2002 EXTRA D NAT COS® from Atochem, silica particles that are surface-treated with a 1 to 2% mineral wax (INCI name: hydrated silica (and) paraffin) such as those sold by Degussa, amorphous silica microspheres, such as those sold under the name SUNSPHERE for example having the reference H-53 by Asahi Glass, and silica microbeads such as those sold under the name SB-700® or SB-150® by Miyoshi, this list not being limiting.

The filler having a blurring effect may be present in the cosmetic composition having a blurring effect in an amount ranging from 0.1 to 20 wt % and especially ranging from 1 wt % to 12 wt % relative to the total weight of the composition, especially between 5 and 10 wt %, for example around 8 wt %.

The term “fluorescent agent” is understood to mean a substance which, under the effect of ultraviolet rays and/or visible light, re-emits into the visible range the portion of light that it has absorbed in the same colour that it reflects naturally. The naturally reflected colour is thus reinforced by the re-emitted colour and appears extremely bright.

Mention may be made, for example, of coloured resins of polyamide and/or of formaldehyde/benzoguanamine and/or of melamine/formaldehyde/sulphonamide, among the coloured aminotriazine/formaldehyde/sulphonamide co-condensates and/or among the metallized polyester flakes and/or mixtures thereof. These fluorescent pigments may also be in the form of aqueous dispersions of fluorescent pigments.

Mention may also be made of the pink-coloured fluorescent aminotriazine/formaldehyde/sulphonamide co-condensate having an average particle size of 3-4 microns, sold under the trade name FIESTA ASTRAL PINK FEX-1 and the blue-coloured fluorescent aminotriazine/formaldehyde/sulphonamide co-condensate having an average particle size of 3-4.5 microns sold under the trade name FIESTA COMET BLUE FTX-60 by Swada or else the benzoguanamine/formaldehyde resin coated with formaldehyde/urea resin and coloured yellow, sold under the trade name FB-205 YELLOW and the benzoguanamine/formaldehyde resin coated with formaldehyde/urea resin and coloured red, sold under the trade name FB-400 ORANGE RED by UK Seung Chemical, the orange-coloured polyamide resin sold under the trade name FLARE 911 ORANGE 4 by Sterling Industrial Colors.

The fluorescent substances are preferably present in the composition in an amount ranging from 0.1 to 20%, preferably from 0.1 to 15%, more preferably from 0.5 to 3% by weight, relative to the total weight of the composition.

When the organic fluorescent substances are white, they are also called optical brighteners.

The optical brightener has the effect of intensifying the radiance and brightening the tones of the cosmetic compositions containing them upon application to the skin.

Among the optical brighteners, mention may more particularly be made of stilbene derivatives, in particular polystyrylstilbenes and triazinestilbenes, coumarin derivatives, in particular hydroxycoumarins and aminocoumarins, oxazole derivatives, benzoxazole, imidazole, triazole, pyrazoline, pyrene derivatives and porphyrin derivatives and/or mixtures thereof.

Such compounds are, for example, available under the trade names TINOPAL SOP® and UVITEX OB® from Ciba Geigy.

The optical brighteners preferentially used are sodium 4,4′-bis(4,6-dianilino-1,3,5-triazin-2-yl)amino]stilbene-2,2′-disulphonate, 2,5-thiophendiylbis(5-tert-butyl-1,3-benzoxazole), disodium distyryl-4,4′-biphenyl sulphonate and/or mixtures thereof.

2. Agents Stimulating the Synthesis of Dermal and/or Epidermal Macromolecules and/or Preventing their Degradation:

Among the active agents that stimulate the dermal macromolecules or that prevent their degradation, mention may be made of those that act:

either on the synthesis of collagen, such as extracts of Centella asiatica, asiaticosides and derivatives; ascorbic acid or vitamin C and its derivatives; synthetic peptides such as iamin, biopeptide CL or palmitoyl oligopeptide sold by Sederma; peptides extracted from plants, such as soybean hydrolysate sold by Coletica under the trade name PHYTOKINE®; rice peptides such as NUTRIPEPTIDE® from Silab, methylsilanol mannuronate such as ALGISIUM C® sold by Exsymol and plant hormones such as auxins and lignans;
or on inhibiting the degradation of collagen, in particular agents acting to inhibit metalloproteinases (MMPs) such as, more particularly, MMPs 1, 2, 3, 9. Mention may be made of: retinoids and derivatives, extracts of Medicago sativa such as VITANOL® from Silab, an extract of Aphanizomenon flos-aquae (cyanophycea) sold under the name LANABLUE® by Atrium Biotechnologies, oligopeptides and lipopeptides, lipoamino acids, malt extracts sold by Coletica under the trade name COLLALIFT®; bilberry or rosemary extracts; lycopene; isoflavones, their derivatives or the plant extracts containing them, in particular extracts of soybean (sold, for example, by Ichimaru Pharcos under the trade name FLAVOSTERONE SB®), red clover, flax, kakkon or sage; litchi extract; dipalmitoyl hydroxyproline sold by Seppic under the name SEPILIFT DPHP®; Baccharis genistelloide or BACCHARINE sold by Silab, a moringa extract such as ARGANYL LS 9781® from Cognis;

or on synthesis of molecules belonging to the elastin family (elastin and fibrillin), such as: retinol and derivatives, in particular retinol palmitate; extracts of Saccharomyces cerivisiae sold by LSN under the trade name CYTOVITIN®; and the extract of Macrocystis pyrifera algae sold by Secma under the trade name KELPADELIE®;

or on inhibiting the degradation of the elastin such as the peptide extract of Pisum sativum seeds sold by LSN under the trade name PARELASTYL®; heparinoids; and the N-acylaminoamide compounds described in Application WO 01/94381 such as {2-[acetyl(3-trifluoromethylphenyl)amino]-3-methylbutyrylamino}acetic acid, otherwise known as N—[N-acetyl-N′-(3-trifluoromethyl)phenylvalyl]glycine or N-acetyl-N-[3-(trifluoro-methyl)phenyl]valylglycine or acetyl trifluoromethyl phenyl valylglycine, or an ester of this with a C₁-C₆ alcohol; an extract of rice peptides such as COLHIBIN® from Pentapharm, or an extract of Phyllanthus emblica such as EMBLICA® from Rona;

or on the synthesis of glycosaminoglycans, such as the product of fermenting milk by Lactobacillus vulgaris, sold by Brooks under the trade name BIOMIN YOGOURTH®; the extract of Padina pavonica brown algae sold by Alban Muller under the trade name HSP3®; the extract of Saccharomyces cerevisiae available especially from Silab under the trade name FIRMALIFT® or from LSN under the trade name CYTOVITIN®; an extract of Laminaria ochroleuca such as LAMINAINE® from Secma; mamaku essence from Lucas Meyer, and a water cress extract (ODRALINE® from Silab);

or on the synthesis of fibronectin, such as the extract of Salina zooplankton sold by Seporga under the trade name GP4G®; the yeast extract available especially from Alban Müller under the trade name DRIELINE®; and palmitoyl pentapeptide sold by Sederma under the trade name MATRIXIL®.

Among the active agents that stimulate the epidermal macromolecules, such as fillagrin and keratins, mention may especially be made of the lupin extract sold by Silab under the trade name STRUCTURINE®; the extract of Fagus sylvatica beech buds sold by Gattefosse under the trade name GATULINE® RC; and the extract of Salina zooplankton sold by Seporga under the trade name GP4G®; copper tripeptide from Procyte; a peptide extract of Voandzeia substerranea such as that sold by Laboratoires Sérobiologiques under the trade name FILLADYN LS 9397®.

Preferably, an additional agent will be used that stimulates the synthesis of dermal and/or epidermal macromolecules and/or prevents their degradation, chosen from agents stimulating the synthesis of glycosaminoglycans, agents inhibiting the degradation of elastin, agents stimulating the synthesis of fibronectin, agents stimulating the synthesis of epidermal macromolecules, and mixtures thereof.

Even more preferentially, an agent will be used that stimulates the synthesis of glycosaminoglycans chosen from an extract of Padina pavonica brown algae, an extract of Saccharomyces cerevisiae, an extract of Laminaria ochroleuca, mamaku essence, a water cress extract and mixtures thereof.

As preferred additional agents that stimulate the synthesis of dermal and/or epidermal macromolecules and/or that prevent their degradation, mention may be made of: biopeptide CL or palmitoyl oligopeptide, an extract of Aphanizomenon flos-aquae (cyanophycea), a malt extract, a moringa extract, an extract of Saccharomyces cerevisiae, a peptide extract of Pisum sativum seeds, {2-[acetyl-(3-trifluoromethylphenyl)amino]-3-methylbutyrylamino}acetic acid, an extract of Padina pavonica brown algae, an extract of Salina zooplankton, a yeast extract, a lupin extract, an extract of Fagus sylvatica beech buds, a peptide extract of Voandzeia substerranea, and mixtures thereof.

3. Agents Stimulating the Proliferation of Fibroblasts or Keratinocytes and/or the Differentiation of the Keratinocytes

The agents stimulating the proliferation of fibroblasts that can be used in the composition according to the invention may, for example, be chosen from plant proteins or polypeptides, especially extracted from soybean (for example a soybean extract sold by LSN under the name ELESERYL SH-VEG 8® or sold by Silab under the trade name RAFFERMINE®); an extract of hydrolysed soybean proteins such as RIDULISSE® from Silab; and plant hormones such as gibberellins and cytokinins.

Preferably, an agent promoting the proliferation and/or differentiation of the keratinocytes will be used.

The agents stimulating the proliferation of the keratinocytes, that can be used in the composition according to the invention, especially comprise adenosine; phloroglucinol; walnut meal extract sold by Gattefosse; and extracts of Solanum tuberosum sold by Sederma; an extract of Larrea divaricata such as CAPISLOW® from Sederma, mixtures of papaya, olive leaves and lemon such as XYLEINE from Vincience, the leaf extract of Hydrangea macrophylla such as AMACHA LIQUID E® from Ichimaru Pharcos, and a yeast extract such as STIMODERM® from CLR.

Among the agents that stimulate the differentiation of the keratinocytes are, for example, minerals such as calcium; a peptide lupin extract such as that sold by Silab under the trade name STRUCTURINE®; sodium β-sitosteryl sulphate such as that sold by Seporga under the trade name PHYTOCOHESINE®; and a water-soluble maize extract such as that sold by Solabia under the trade name PHYTOVITYL®; a peptide extract of Voandzeia substerranea such as that sold by Laboratoires Sérobiologiques under the trade name FILLADYN LS 9397®; and lignans such as secoisolariciresinol, retinol and its esters including retinyl palmitate.

As agents stimulating the proliferation and/or differentiation of the keratinocytes, mention may also be made of oestrogens such as oestradiol and homologues; and cytokines.

As preferred additional agents that stimulate the proliferation of fibroblasts or keratinocytes and/or the differentiation of the keratinocytes, mention will be made of a lupin extract, an extract of Larrea divaricata, a yeast extract and mixtures thereof.

4. Agents Favouring the Skin Barrier Function

As an agent having a skin barrier restructuring effect, mention may be made of an extract of Thermus thermophilus such as VENUCEANE® from Sederma, an extract of wild yam (Dioscorea villosa) rhizome such as ACTIGEN Y® from Active Organics, plankton extracts such as OMEGA PLANKTON® from Secma, yeast extracts such as RELIPIDIUM® from Coletica, a chestnut extract such as RECOVERINE® from Silab, a cedar bud extract such as GATULINE ZEN® from Gattefosse, PHYTOSPHINGOSINE SLC from Degussa, AQUAXYL® from Seppic, LIPIDESSENCE® from Coletica.

Mention may also be especially made of ceramides and derivatives, compounds based on sphingoids, glycosphingolipids, phospholipids, cholesterol and its derivatives, phytosterols, essential fatty acids, diacyl glycerol, 4-chromanon and chromon derivatives, petroleum jelly, lanolin, shea butter, cocoa butter, lanolin, etc.

As preferred additional agents favouring the skin barrier function, mention will be made of an extract of Thermus thermophilus, an extract of wild yam (Dioscorea villosa) rhizome, a yeast extract, a chestnut extract, a cedar bud extract and mixtures thereof.

5. Anti-Glycating Agents

As anti-glycating agents, mention may especially be made of erogthioneine, dihydroxystilbenes and their derivatives, hydroxyimides and derivatives such as those cited in Application WO 2005/054192, ANTIGLYSKIN® from Silab, AMADORINE® from Solabia, wine extracts from FIS, NUTRALIP® from Labochim, AGLYCAL LS 8777® from LSN, a black tea extract, KOMBUCHKA from Sederma, lipoic or thioctic acid, or bilberry (Vaccinium myrtillus) extracts.

As preferred anti-glycating agents, mention will be made of bilberry (Vaccinium myrtillus) extracts.

6. Agents that Favour Maturing of the Horny Envelope

It will be possible to use, in the compositions of the invention, agents that are involved in maturing of the horny envelope which deteriorates with age and leads to a reduction of transglutaminase activity. Mention may be made, for example, of urea and its derivatives and in particular HYDROVANCE® from National Starch and the other active agents mentioned in L'Oréal Application FR 2 877 220 (unpublished).

7. NO-Synthase Inhibitors

It is also possible to combine agents having an NO-synthase inhibiting action such as PCOs (procyanidolic oligomers), grape leucocyanidins (LEUCOSELECT from Indena, grape-cake extract from Hansen, or from Euromed), extracts of Olea europea such as those from Vinyals, extracts of Ginkgo biloba (EUROL BT from Biologiax Technologia), extracts of grape seeds, of hawthorn such as those from Berkem and of pine bark such as those from Euromed.

8. Peripheral Benzodiazepine Receptor (PBR) Antagonists

Mention may be made, for example, of 1-(2-chlorophenyl)-N-(1-methylpropyl)-3-isoquinolinecarboxamide; the compounds described in Applications WO 03/030937, WO 03/068753, and pyridazino[4,5-b]indole-1-acetamide derivatives of general formula (VII) as described in document WO 00/44384.

9. Agents Increasing the Activity of the Sebaceous Gland

Mention may be made, for example, of methyl dihydrojasmonate, hecogenin, hedione, O-linoleyl-6-D-glucose and mixtures thereof.

10. Skin-Relaxing or Dermo-Decontracting Agents

As examples, mention may be made of manganese gluconate and other salts, adenosine, alverine citrate and its salts, glycine, an extract of Iris pallida, a hexapeptide (ARGERILINE R from Lipotec) or sapogenins such as wild yam and the carbonylated amines described in Application EP 1 484 052. As an example of sapogenins, mention may be made of those described in Patent Application WO 02/47650, in particular wild yam, the diosgenin extracted especially from Dioscorea opposita or any extracts containing, naturally or after treatment, one or more sapogenins (wild yam rhizome, agave leaf that contains hecogenin and tigogenin, extract of Liliaceae and more particularly the Yucca or Smilax containing smilagenin and sarsapogenin or the sarsaparilla root) or ACTIGEN Y from Active Organics.

Mention may also be made of DMAE (dimethyl MEA), extracts of sea fennel, Montpellier cistus, helichrysum, anise, paracress, an extract of Acmella oleracea such as GATULINE EXPRESSION from Gattefosse.

As preferred additional skin-relaxing agents, mention will be made of manganese gluconate, glycine and alverine.

11. Agents Favouring Skin Microcirculation

It is possible to combine, in the compositions of the invention, agents acting on the skin microcirculation in order to prevent dulling of the skin tone and/or formation of bags, such as for example, a black tea extract such as KOMBUCHKA from Sederma, pycnogenol, manganese gluconate (GIVOBIO GMn from Seppic), VISNADINE from Indena, a lupin extract (ECLALINE from Silab), EPALINE 100 from Laboratoires Carilene, a Seville orange flower extract (REMODULINE from Ilab), vitamin P and its derivatives such as PERMETHOL from Sochibios and other extracts (of ruscus, horse chestnut, ivy, ginseng, sweet clover, etc.) and also caffeine, escin, hespertine laurate, dextran sulphate, nicotinate and derivatives, lysine and derivatives (such as ASPARLYNE from Solabia), etc.

As preferred additional agents favouring skin microcirculation, mention will be made of KOMBUCHKA, manganese gluconate and vitamin P and its derivatives.

12. Agents Stimulating the Cell Energy Metabolism

It is also possible to combine active agents that stimulate the energy metabolism which is found to slow down during ageing, among which mention may be made of biotin, an extract of Saccharomyces cerevisiae such as PHOSPHOVITAL® from Sederma, PHYSIOGENYL® from Solabia, a mixture of zinc, copper and manganese gluconate such as SEPITONIC M3® from Seppic.

13. Tightening Agents

The term “tightening agent” which can be used in the compositions of the invention, is understood to mean compounds capable of having a tightening effect, that is to say which can tighten the skin.

The tightening effect may be characterized by an in vitro retraction test. This test consists in quantifying in vitro the tightening ability of a material deposited on an elastomeric substrate having a modulus of around 20 MPa and a thickness of 100 μm. The solution containing the tightening agent at a concentration of 7 wt % is therefore deposited (30 μl) onto a rectangular (10×40 mm) elastomeric test piece. After drying for 3-4 h at 22±3° C. and 40±10% relative humidity RH, the tension exerted by this deposit on the substrate and consequently the tightening potential is directly linked to the decrease in the width at the centre of the test piece. The tightening effect (TE) can then be quantified in the following manner:

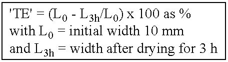

Generally, the term “tightening agent” is understood to mean any compound producing, at a concentration of 7 wt % in water, or in any physiologically acceptable medium, a retraction of more than 15% in the previously described test.

The tightening agent may be chosen from:

a) plant or animal proteins and their hydrolysates;

b) polysaccharides of plant origin;

c) mixed silicates;

d) colloidal particles of inorganic fillers;

e) synthetic polymers;

and mixtures thereof.

Mention may especially be made of:

(1) synthetic polymers, such as polyurethane latices or acrylic/silicone latices, in particular those described in Patent Application EP 1 038 519, such as a propylthio(polymethyl acrylate), propylthio(polymethyl methacrylate) and propylthio(polymethacrylic acid) grafted polydimethylsiloxane or else a propylthio(polyisobutyl methacrylate) and propylthio(poly-methacrylic acid) grafted polydimethylsiloxane. Such grafted silicone polymers are especially sold by 3M under the trade names VS 80, VS 70 or LO21;

(2) polymers of natural origin, especially (a) polyholosides, for example (i) in the form of starch derived especially from rice, maize, potato, cassava, peas, wheat, oats, etc. or (ii) in the form of carrageenans, alginates, agars, gellans, cellulose polymers and pectins, advantageously as an aqueous dispersion of gel microparticles, and (b) latices composed of shellac resin, gum sandarac, dammars, elemis, copals, cellulose derivatives, and mixtures thereof;

(3) proteins and hydrolysates of plant proteins, in particular of maize, rye, wheat, buck wheat, sesame, spelt, pea, bean, lentil, soybean and lupin,

(4) mixed silicates, especially phyllosilicates and in particular laponites;

(5) wax microparticles, for example chosen from carnauba, candelilla or alfa waxes; and

(6) colloidal particles of inorganic fillers having a number-average diameter between 0.1 and 100 nm, preferably between 3 and 30 nm, and chosen, for example, from: silica, silica/alumina composites, cerium oxide, zirconium oxide, alumina, calcium carbonate, barium sulphate, calcium sulphate, zinc oxide and titanium dioxide.

As another additional anti-ageing agent, mention may be made of DHEA and its derivatives, adenosine and its derivatives especially disodium adenosine monophosphate, boswellic acid, rosemary extracts, carotenoids (β-carotene, zeaxanthin, lutein, cysteic acid, 5-(n-octanoyl)salicylic acid, gentisic acid and its derivatives, lignans and their glycosyl derivatives, flavonoids and flavosterones, copper derivatives, jasmonic acid, resveratrol and its derivatives, retinol and its derivatives.

It could be advantageous to also add to the composition used in the process according to the invention, agents promoting the naturally pink colouring of the skin, such as:
a self-tanning agent, that is to say an agent which, when applied to the skin, especially to the face, gives a tanning effect that is more or less similar in appearance to that which may result from prolonged exposure to sunlight (natural tan) or under a UV lamp;
an additional colouring agent, that is to say any compound having a particular affinity for the skin that allows it to give the skin a lasting, non-covering (i.e. that does not have a tendency to opacify the skin) coloration and that is not removed either with water or using a solvent, and that withstands both rubbing and washing with a solution containing surfactants. Such a lasting coloration is therefore distinguished from the superficial and transient coloration provided, for example, by a makeup pigment;
   and mixtures thereof.

As examples of self-tanning agents, mention may be made of:

dihydroxyacetone (DHA);

erythrulose; and

the combination of a catalytic system formed from:
manganese and/or zinc salts and oxides; and
alkali-metal and/or alkaline-earth-metal hydrogencarbonates.

The self-tanning agents are generally chosen from moncarbonylated or polycarbonylated compounds such as, for example, isatin, alloxan, ninhydrin, glyceraldehydes, mesotartric aldehyde, glutaraldehyde, erythrulose, pyrazolin-4,5-dione derivatives as described in Patent Application FR 2 466 492 and WO 97/35842, dihydroxyacetone (DHA), 4,4-dihydroxypyrazolin-5-one derivatives as described in Patent Application EP 903 342. Preferably, DHA will be used.

DHA may be used in free and/or encapsulated form, for example in lipid vesicles such as liposomes, especially described in Application WO 97/25970.

Generally, the self-tanning agent is present in an amount ranging from 0.01 to 20 wt %, and preferably in an amount between 0.1 and 10 wt % of the total weight of the composition.

As additional colouring agents, mention may be made, for example, of plant extracts such as, for example, the sorghum extracts obtained from the whole plant, the stems, the seeds or the leaves of the genus Sorghum. The preferred species of Sorghum are chosen from Sorghum bicolor, Sorghum caudatum, Sorghum nervosum, Sorghum durra, Sorghum vulgare and Sorghum plants in combination with Colletotrichum graminicola such as those described in Patent Application FR 0 200 251.

It is also possible to use other colorants that enable the colour produced by the self-tanning agent to be modified.

These colorants may be chosen from direct synthetic or natural colorants.

These colorants may be chosen, for example, from red or orange colorants of the fluorane type such as those described in Patent Application FR 2 840 806. Mention may be made, for example, of the following colorants:

tetrabromofluorescein or eosin known under the CTFA name: CI 45380 or Red 21;

phloxine B known under the CTFA name: CI 45410 or Red 27;

diiodofluorescein known under the CFTA name: CI 45425 or Orange 10;

dibromofluorescein known under the CTFA name: CI 45370 or Orange 5;

the sodium salt of tetrabromofluorescein known under the CTFA name: CI 45380 (Na salt) or Red 22;

the sodium salt of phloxine B known under the CTFA name: CI 45410 (Na salt) or Red 28;

the sodium salt of diiodofluorescein known under the CTFA name: CI 145425 (Na salt) or Orange 11;

erythrosine known under the CTFA name: CI 45430 or Acid Red 51; and

phloxine known under the CTFA name: CI 45405 or Acid Red 98.

These colorants may also be chosen from anthraquinones, caramel, carmine, carbon black, azulene blues, methoxalene, trioxalene, guajazulene, chamuzulene, rose bengal, cosine 10B, cyanosine, and daphnin.

These colorants may also be chosen from indole derivatives such as monohydroxyindoles as described in Patent FR 2 651 126 (i.e. 4-, 5-, 6- or 7-hydroxyindole) or dihydroxyindoles as described in Patent EP-B-0 425 324 (i.e. 5,6-dihydroxyindole, 2-methyl-5,6-dihydroxyindole, 3-methyl-5,6-dihydroxyindole, 2,3-dimethyl-5,6-dihydroxyindole).

The additional active agent(s) and/or ingredient(s) used in the composition according to the invention may represent from 0.0001 to 20 wt %, preferably from 0.01 to 10 wt % and best from 0.01 to 1 wt % relative to the total weight of the composition.

Cosmetic Assembly

According to another aspect, the invention also relates to a cosmetic assembly comprising:
i) a container delimiting at least one compartment, said container being closed by means of a closing member; and
ii) a composition as described previously placed inside said compartment.

The container may be any suitable form. It may especially be in the form of a bottle, tube, pot, jar, case, sachet or carton.

The closing member may be in the form of a removable stopper, a lid, a cap, a tear-off strip, or a capsule, especially of the type comprising a body attached to the container and a cover cap articulated on the body. It may also be in the form of a member for selectively closing the container, especially a pump, a valve or a flap valve.

The container may be combined with an applicator. The applicator may be in the form of a brush, as described, for example, in Patent FR 2 722 380. The applicator may be in the form of a foam or elastomer block, a felt or a spatula. The applicator may be free (powder puff or sponge) or securely fastened to a wand borne by the closing member, as described, for example, in Patent U.S. Pat. No. 5,492,426. The applicator may be securely fastened to the container, as described, for example, in Patent FR 2 761 959.

The product may be contained directly in the container, or indirectly. By way of example, the product may be placed on an impregnated support, especially in the form of a wipe or a pad, and placed (individually or in plurality) in a box or in a sachet. Such a support incorporating the product is described, for example, in Application WO 01/03538.

The closing member may be coupled to the container by screwing. Alternatively, the coupling between the closing member and the container is done other than by screwing, especially via a bayonet mechanism, by snap-fastening, gripping, welding, bonding or by magnetic attraction. The term “snap-fastening” is understood to mean, in particular, any system that involves crossing a band or strip of material via elastic deformation of one portion, especially of the closing member, followed by return to the elastically unconstrained position of said portion after crossing of the band or strip.

The container may be at least partially produced from a thermoplastic. As examples of thermoplastic materials, mention may be made of polypropylene or polyethylene.

Alternatively, the container is produced from a non-thermoplastic material, especially glass or metal (or alloy).

The container may have rigid walls or deformable walls, especially in the form of a tube or a tubular bottle.

The container may comprise means intended for initiating or facilitating the distribution of the composition. By way of example, the container may have deformable walls so as to allow the composition to exit in response to a positive pressure inside the container, this positive pressure being caused by elastic (or non-elastic) squeezing of the walls of the container.

The container may be formed from a carton having a base that delimits at least one housing containing the composition, and a lid, especially articulated onto the base, and capable of at least partially covering said base. Such a carton is described, for example, in Application WO 03/018423 or in Patent FR 2 791 042.

The container may be equipped with a squeezing member placed in the vicinity of the container opening. Such a squeezing member makes it possible to wipe the applicator and optionally the wand to which it may be attached. Such a squeezing member is described, for example, in Patent FR 2 792 618.

The content of the aforementioned patents or patent applications is incorporated by reference into the present application.

According to a particular mode of the invention, the assembly may comprise:
a composition A containing at least one C-glycoside derivative and optionally an ingredient favouring its solubilization and/or its stabilization and/or its activity; and
a composition B, packaged separately to the composition A, comprising at least one additional cosmetic or dermatological active ingredient.

In this case, the compositions A and B may be packaged separately inside two compartments, formed either from two separate containers, or inside a single device.

The term “single device” is understood to mean a device for which the two compartments are securely fastened to each other. Such a device may be obtained by a process of one-piece moulding via two compartments, especially from a thermoplastic. It may also result from any form of assembly, especially by bonding, welding or else snap-fastening.

According to a first embodiment, the two containers are independent of one another. Such containers may be in various forms. They may especially be tubes, bottles or cans.

One and/or the other of the containers may be topped with a manually activated pump that is topped with a pushbutton for activating the pump and distributing the composition via at least one distribution orifice.

Alternatively, one and/or the other of the containers is pressurized, especially by means of a propellant, in particular a propellent gas. In this case, the container(s) is (are) equipped with a valve topped by a pushbutton equipped with a nozzle or any other dispersal means for distributing the product.

The propellant may be mixed with the composition to be distributed or may be separate from it, in particular via a piston capable of sliding inside the container, or via the flexible walls of a pouch inside which the composition is placed.

The containers may be formed from various materials: plastic, glass or metal.

Alternatively again, the two compartments are formed from two concentric compartments formed inside a tube, and are topped with an airless pump equipped with a pushbutton having one or two distribution orifices. A piston is provided inside the tube which gradually rises in the direction of the pump as the compositions are removed from inside the containers. Such distribution methods are used especially for distributing toothpastes.

The invention also relates to a cosmetic method for caring for and/or cleansing and/or making up the skin or its appendages that comprises applying a composition as defined previously to the skin or its appendages.

The term “appendages” is understood according to the invention to mean the skin, nails, eyelashes and/or body hair, and head hair.

Preferably, this will be a composition for the skin.

In particular, the method according to the invention is intended to promote lightening and/or depigmentation of the skin and/or to homogenize the complexion and/or to smooth the microrelief (to reduce wrinkles and fine lines), and/or to prevent or combat the signs of skin ageing.

According to another embodiment, it is intended to reduce the cuteneous imperfections of greasy skin, in particular to matify the skin.

The term “cutaneous imperfections of greasy skin” is understood, in particular according to the invention, to mean aesthetic disorders such as shiny skin, a poorer staying power for makeup, a thick skin grain generally associated with a desquamating defect, a skin whose follicular orifices are dilated or filled with minute horny spicules, or even with comedones or blackheads (resulting however more from a retention phenomenon that from an increase in excretion).

The term “matify” is understood to mean to make the skin visibly more matt, less shiny. The matifying effect of the composition may especially be evaluated using a gonioreflectometer, by measuring the ratio R between the specular reflection and the diffuse reflection. An R value less than or equal to 2 generally indicates a matifying effect.

In particular, the composition is applied to the areas of the face or the forehead having shiny skin.

According to another application of the invention, the method is intended to reduce the cutaneous imperfections linked to ageing, in particular to actinic ageing.

The term “cutaneous imperfections linked to skin ageing”, in particular to actinic ageing, is understood to mean, in particular, a loss of firmness and/or elasticity and/or tone and/or suppleness of the skin, the formation of wrinkles and fine lines, a dull appearance of the complexion, appearance of darkening and/or yellowing of the skin, and/or appearance of senescence spots or “age spots”.

It will especially be intended for people with mature, even very mature, skin.

The term “mature skin” is understood according to the invention to mean, in particular, people who are at least 40 years old.

The term “very mature skin” is understood according to the invention to mean, in particular, people who are at least 50 years old, in particular at least 60 years old, even 65 years old.

According to a particular mode of the invention, the method is intended for the care of people having dark skin (especially skin of phototype IV to VI).

According to another mode of the invention, the method is intended for the care of people having light skin (especially skin of phototype I to III).

What is claimed is:

1. A cosmetic and/or dermatological composition comprising, in a physiologically acceptable medium, at least one C-glycoside derivative and at least one additional cosmetic and/or dermatological ingredient and/or active agent.

2. A cosmetic and or dermatological composition according to claim 1, wherein said at least one additional cosmetic agent is an agent acting on skin microcirculation in order to prevent dulling of the skin tone and/or formation of bags and is chosen from a black tea extract, pycnogenol, manganese gluconate, a lupin extract, a Seville orange flower extract, vitamin P and its derivatives, extract of ruscus, extract of horse chestnut, extract of ivy, extract of ginseng, extract of sweet clover, caffeine, escin, hespertine laurate, dextran sulphate, nicotinate and derivatives, and lysine and derivatives.

3. A cosmetic and/or dermatological composition comprising, in a physiologically acceptable medium, at least two separate compositions:
at least one first composition comprising at least one C-glycoside derivative and at least one second composition comprising at least one additional cosmetic and/or dermatological ingredient and/or active agent.

4. A cosmetic treatment method for caring for, cleansing and/or making up the skin and/or its appendages comprising applying to said skin or its appendages a cosmetic and/or dermatological composition comprising, in a physiologically acceptable medium, at least one C-glycoside derivative and at least one additional cosmetic and/or dermatological ingredient and/or active agent.
